(19) United States
(12) Patent Application Publication
(10) Pub. No.: US 2019/0224675 A1
(43) Pub. Date: Jul. 25, 2019

(54) FLUIDIC SYSTEM AND RELATED METHODS

(71) Applicant: ArcherDX, Inc., Boulder, CO (US)

(72) Inventors: Joshua Stahl, Boulder, CO (US); Jason Myers, Boulder, CO (US)

(73) Assignee: ArcherDX, Inc., Boulder, CO (US)

(21) Appl. No.: 16/336,342

(22) PCT Filed: Sep. 22, 2017
(86) PCT No.: PCT/US2017/053106

§ 371 Date: Mar. 25, 2019

Related U.S. Application Data
(63) Continuation of application No. PCT/US2017/051924, filed on Sep. 15, 2017.
Application No. PCT/US2017/051924 is a continuation of application No. PCT/US2017/051927, filed on Sep. 15, 2017.
(60) Provisional application No. 62/398,841, filed on Sep. 23, 2016.
(60) Provisional application No. 62/399,152, filed on Sep. 23, 2016.
(60) Provisional application No. 62/399,157, filed on Sep. 23, 2016.
(60) Provisional application No. 62/399,184, filed on Sep. 23, 2016.
(60) Provisional application No. 62/399,195, filed on Sep. 23, 2016.
(60) Provisional application No. 62/399,205, filed on Sep. 23, 2016.
(60) Provisional application No. 62/399,211, filed on Sep. 23, 2016.
(60) Provisional application No. 62/399,219, filed on Sep. 23, 2016.

(51) Int. Cl. B01L 3/00; G01N 35/02; C12Q 1/686

(52) U.S. Cl. CPC B01L 3/502738; G01N 35/026; C12Q 1/686; B01L 2200/027; B01L 2300/0883; B01L 2300/041; B01L 2300/12; B01L 2300/02; B01L 2200/16

(57) ABSTRACT

Fluidic systems including cartridges with modular components (cassettes) and/or microfluidic channels for performing chemical and/or biological analyses are provided. The systems described herein include a cartridge comprising, in some embodiments, a frame, one or more cassettes which may be inserted into the frame, and a channel system for transporting fluids. In certain embodiments, the one or more cassettes comprise one or more reservoirs or vessels configured to contain and/or receive a fluid (e.g., a stored reagent, a sample). In some cases, the stored reagent may include one or more lyospheres. The systems and methods described herein may be useful for performing chemical and/or biological reactions including polymerase chain reactions (PCR) such as those performed within a laboratory, clinical (e.g., hospital), or research setting.

### RELATED APPLICATIONS

This application claims priority under 35 U.S.C. § 119(e) to U.S. Provisional Patent Application Nos. 62/398,841, 62/399,152, 62/399,157, 62/399,184, 62/399,195, 62/399,205, 62/399,211, and 62/399,219, each of which was filed on Sep. 23, 2016, and claims priority under 35 U.S.C. §§ 120 and 365(c) to PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and to PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which applications are hereby incorporated by reference.

### TECHNICAL FIELD

The present invention generally relates to systems and related methods for automated processing of molecules (e.g., nucleic acids). The present invention also generally relates to fluidic systems and related methods.

### BACKGROUND

Numerous approaches for processing nucleic acids have been developed. Such methods often included multiple enzymatic, purification, and preparative steps that make them laborious and prone to error, including errors associated with contamination, systematic user errors, and process biases. As a result, it is often difficult to execute such processes reliably and reproducibly, particularly when the processes are being conducted commercially, e.g., in a multiplex or high-throughput context.

### SUMMARY

The present invention generally relates to systems and related methods for processing nucleic acids. In some embodiments, the system comprises cartridges including cassettes and/or microfluidic channels that facilitate automated processing of nucleic acids, including automated nucleic acid library preparations. In some embodiments, systems and related methods are provided for automated processing of nucleic acids to produces material for next generation sequenceing and/or other downstream analytical techniques. The present invention also generally relates to fluidic systems and related methods.

In one set of embodiments, a series of cartridges are provided. In one embodiment, a cartridge comprises a frame comprising a first opening constructed and arranged to house a first cassette and a second opening to house a second cassette; and a channel system adjacent to and non-integral to the frame, wherein the channel system comprises at least one microfluidic channel, wherein the cassette is configured to allow fluidic communication between the first cassette and the at least one channel of the channel system and/or the second cassette and the at least one channel of the channel system upon insertion of the first and/or second cassette into the frame, respectively.

In another embodiment, a cartridge comprises a frame comprising a first opening constructed and arranged to house a first cassette and a second opening to house a second cassette; a channel system adjacent to the frame, wherein the channel system comprises at least one microfluidic channel; and an first set of vessels, wherein at least a portion of the first set of vessels contains at least one lyosphere disposed therein, wherein the cartridge is configured to allow fluidic communication between at least one vessel and at least one microfluidic channel of the channel system.

In another embodiment, a cartridge comprises a frame comprising a first opening and a second opening; a channel system adjacent to the frame, wherein the channel system comprises at least one microfluidic channel, a first cassette configured to be positioned in the first opening of the frame, wherein the first cassette comprises a first set of vessels; a second cassette configured to be positioned in the second opening of the frame, wherein the first cassette comprises a first set of vessels; and a stored liquid reagent contained in at least one of the first set of vessels of the first cassette, wherein the vessel(s) containing the stored liquid reagent is/are sealed so as to reduce or prevent evaporation of the stored liquid reagent.

In another embodiment, a cartridge comprises a channel system, wherein the channel system comprises at least one microfluidic channel; a first cassette comprising a first set of vessels; a first set of stored reagents for conducting a first PCR reaction contained in the first set of vessels; a second cassette comprising a second set of vessels; and a second set of stored reagents for conducting a second PCR reaction contained in the second set of vessels, wherein the cartridge is constructed and arranged to allow conduction of the first and second PCR reactions in parallel, and to allow fluid communication between the channel system and at least one of the first and second cassettes during conduction of the first and/or second PCR reactions, respectively.

In another embodiment, a cartridge comprises a common microfluidic channel; a sample inlet connected to a sample channel; a first vessel connected to a first vessel channel; a second vessel connected to a second vessel channel; a first valve; and a second valve; wherein each of the common channel, sample channel, first vessel channel and second vessel channel extend from the first valve, and wherein the common microfluidic channel is positioned between the first valve and the second valve.

In another embodiment, a cartridge comprises a first set of vessels; a first valve; a first set of vessel channels connected to the first set of vessels, wherein each of the channels from the first set of vessel channels is connected to the first valve; a second set of vessel channels; and a common microfluidic channel positioned between the first set of vessel channels and the second set of vessel channels.

In another set of embodiments, a series of methods are provided. In one embodiment, a method comprises flowing, in a first direction, a first fluid in a common microfluidic channel; flowing at least a portion of the first fluid in the common microfluidic channel in a second direction, wherein the second direction is opposite the first direction; flowing at least a portion of the first fluid into a first vessel via a first vessel channel; flowing at least a portion of the first fluid from the first vessel to the common microfluidic channel; and flowing at least a portion of the first fluid from the common channel to a second vessel via a second vessel channel.

In one embodiment, a method comprises flowing a first fluid in a common microfluidic channel; flowing a portion of the first fluid into a first vessel; flowing a portion of the first fluid into a waste vessel; performing a chemical and/or biological reaction in the first vessel to form a second fluid; flowing a portion of the second fluid from the first vessel to the common microfluidic channel; and flowing a portion of the second fluid into the waste vessel.

In one embodiment, a method comprises flowing a first fluid into a common microfluidic channel; actuating a valve such that the common microfluidic channel is in fluidic communication with a first vessel channel; flowing at least a portion of the first fluid into the first vessel channel; introducing a second fluid into the common microfluidic channel, wherein the second fluid is immiscible with the first fluid; flowing at least a portion of the second fluid from the common microfluidic channel into the first vessel channel; and flowing a controlled volume of the first fluid into a first vessel connected to the first vessel channel during flow of the second in the first vessel channel.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic drawing of a nucleic acid library preparation workflow;

FIG. 2A is a drawing of a system for automated nucleic acid library preparation using a microfluidic cartridge;

FIG. 2B is a drawing showing internal components of a system for automated nucleic acid library preparation using a microfluidic cartridge;

FIG. 3 is a perspective view of a microfluidic cartridge bay assembly;

FIG. 4A is a top view of a microfluidic cartridge carrier assembly;

FIG. 4B is a perspective view of a microfluidic cartridge;

FIG. 5 is an exploded view of a microfluidic cartridge;

FIG. 6A is a side view of a module being inserted into a cartridge;

FIG. 6B is a side view of a module inserted into a cartridge;

FIG. 6C is a side view of two modules being inserted into a cartridge;

FIG. 7 is a side view of a module including a series of vessels;

FIG. 8 is a side view of a module including a series of vessels containing lyospheres;

FIG. 9 is a top view of a channel system including a common microfluidic channel, series of vessels, and a series of vessel channels;

FIG. 10 is a top view of a channel system including a rotary valve connected to fluidic channels;

FIG. 11 is a top view of a channel system including a series of fluidic channels connected to vessels and other components;

FIG. 12 is a top view of a channel system including fluid flowing towards a rotary valve;

FIG. 13 is a top view of the channel system shown in FIG. 12 including a fluid flowing in a common microfluidic channel;

FIG. 14 is a top view of the channel system shown in FIG. 12 including a fluid in a first vessel;

FIG. 15 is a top view of the channel system shown in FIG. 12 including a fluid flowing in a common microfluidic channel;

FIG. 16 is a top view of the channel system shown in FIG. 12 including a fluid in a second vessel;

FIG. 17 is a top view of a channel system showing a fluid in a common microfluidic channel;

FIG. 18 is a top view of the channel system shown in FIG. 17 showing a fluid being transported from a common microfluidic channel to a first vessel channel;

FIG. 19 is a top view of the channel system shown in FIG. 17 showing an immiscible fluid being used to partition the fluid in a common microfluidic channel;

FIG. 20 is a top view of the channel system shown in FIG. 17 showing an immiscible fluid being used to push a portion of the partitioned fluid towards a first vessel;

FIG. 21 is a top view of the channel system shown in FIG. 17 showing the partitioned fluid in a first vessel;

FIG. 22 is a perspective view showing layers of a microfluidic cartridge;

FIG. 23 is another perspective view showing layers of a microfluidic cartridge;

FIG. 24 is a perspective view showing various layers of a microfluidic cartridge;

FIG. 25 is a top view of a channel system including various valves; and

FIG. 26 is another top view of a channel system including various valves.

### DETAILED DESCRIPTION

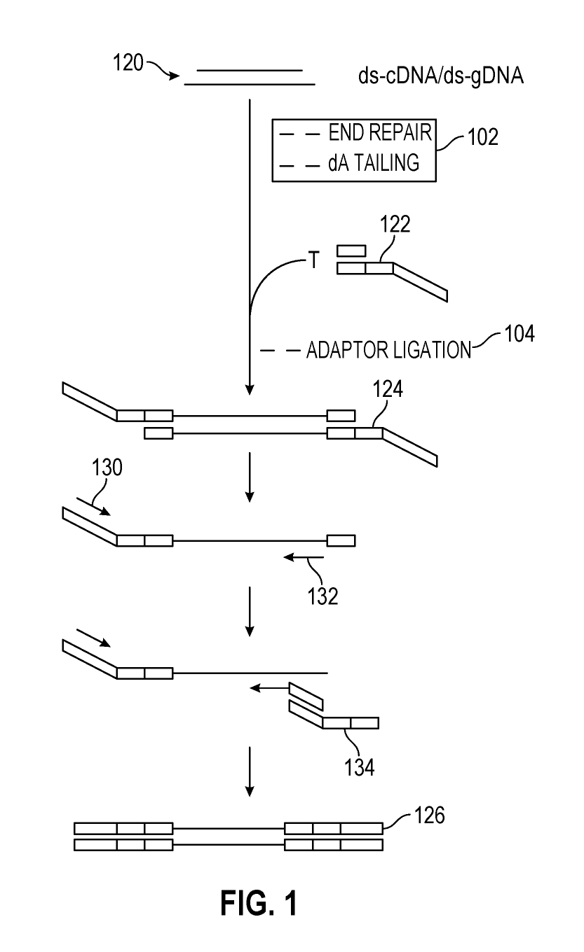

Systems including cartridges with modular components (cassettes) and/or microfluidic channels for processing nucleic acids are generally provided. In some embodiments, systems and related methods are provided for automated processing of nucleic acids to produce material for next generation sequencing and/or other downstream analytical techniques. In some embodiments, systems described herein include a cartridge comprising, a frame, one or more cassettes which may be inserted into the frame, and a channel system for transporting fluids. In certain embodiments, the one or more cassettes comprise one or more reservoirs or vessels configured to contain and/or receive a fluid (e.g., a stored reagent, a sample). In some cases, the stored reagent may include one or more lyospheres. The systems and methods described herein may be useful for performing chemical and/or biological reactions including reactions for nucleic acid processing, including polymerase chain reactions (PCR). In some embodiments, systems and methods provided herein may be used for processing nucleic acids as depicted in FIG. 1. For example, in some embodiments, the nucleic acid preparation methods depicted in FIG. 1, which are described in greater detail herein, may be conducted in a multiplex fashion with multiple different (e.g., up to 8 different) samples being processed in parallel in an automated fashion. Such systems and methods may be implemented within a laboratory, clinical (e.g., hospital), or research setting.

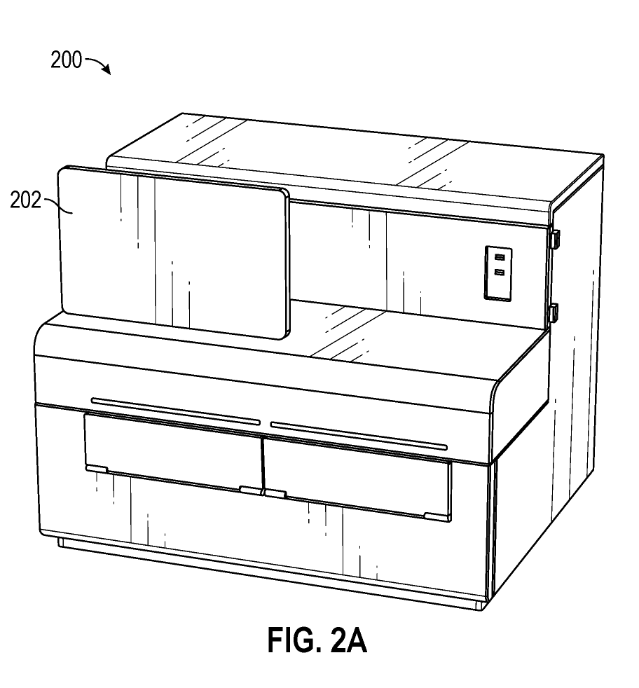

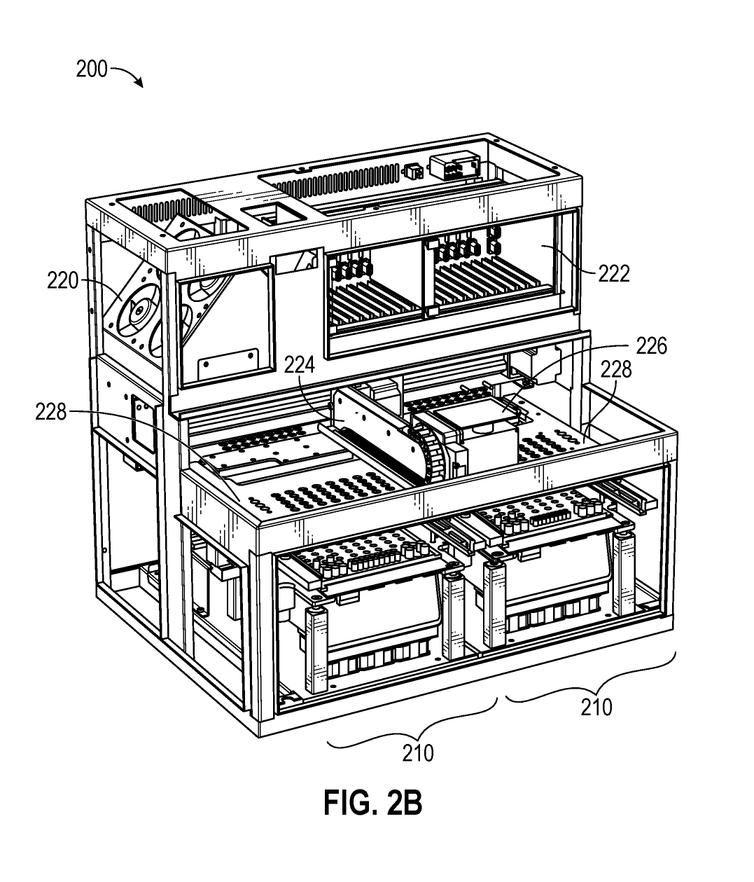

In some embodiments, systems provided herein may be used for next generation sequencing (NGS) sample preparation (e.g., library sample preparation). In some embodiments, systems provided herein may be used for sample quality control. FIGS. 2A and 2B depict an example system 200 which serves as a laboratory bench top instrument which utilizes a number of disposable cassettes, primer cassettes, and bulk fluid cassettes. In some embodiments, this system is suitable for use on a standard laboratory workbench.

In some embodiments, a system may have a touch screen interface (e.g., as depicted in the exemplary system of FIG. 2A comprising a touch screen interface 202). In some embodiments, the interface displays the status of each of the one or more cartridge bays with “estimated time to complete”, “current process step”, or other indicators. In some embodiments, a log file or report may be created for each of the one or more cartridges. In some embodiments, the log file or report may be saved on the instrument. In some embodiments, a text file or output may be sent from the instrument, e.g., for a date range of cartridges processed or for a cartridge with a particular serial number.

In some embodiments, systems provided herein may comprise one or more cartridge bays (e.g., two, as depicted in the exemplary system of FIG. 2B comprising two cartridge bays 210), capable of receiving one or more nucleic acid preparation cartridges. In some embodiments, a space above the cartridge bay(s) is reserved for an XY positioner 224 to move an optics module 226 (and/or a barcode scanner, e.g., a 2-D barcode scanner) above lids 228 (e.g., heated lids) of each cartridge bay. In some embodiments, the system comprises an electronics module 222 that drives optics module 226 and XY positioner 224. In some embodiments, XY positioner 224 will position optics module 226 such that it can excite materials (e.g., fluorophores) in the vessel and collect the emitted fluorescent light. In some embodiments, this will occur through holes placed in the lid (e.g., heated lid) over each vessel. In some embodiments, a barcode scanner will confirm that appropriate cartridge and primer cassettes have been inserted in the system. In some embodiments, optics module 226 will collect light signals from each cartridge in each cartridge bay, as needed, during processing of a sample, e.g., during amplification of a nucleic acid to detect the level of the amplified nucleic acid. In some embodiments, the systems described herein comprise elements that assist in temperature regulation of components within the system, such as one or more fans or fan assemblies (e.g., the fan assembly 220 depicted in FIG. 2B).

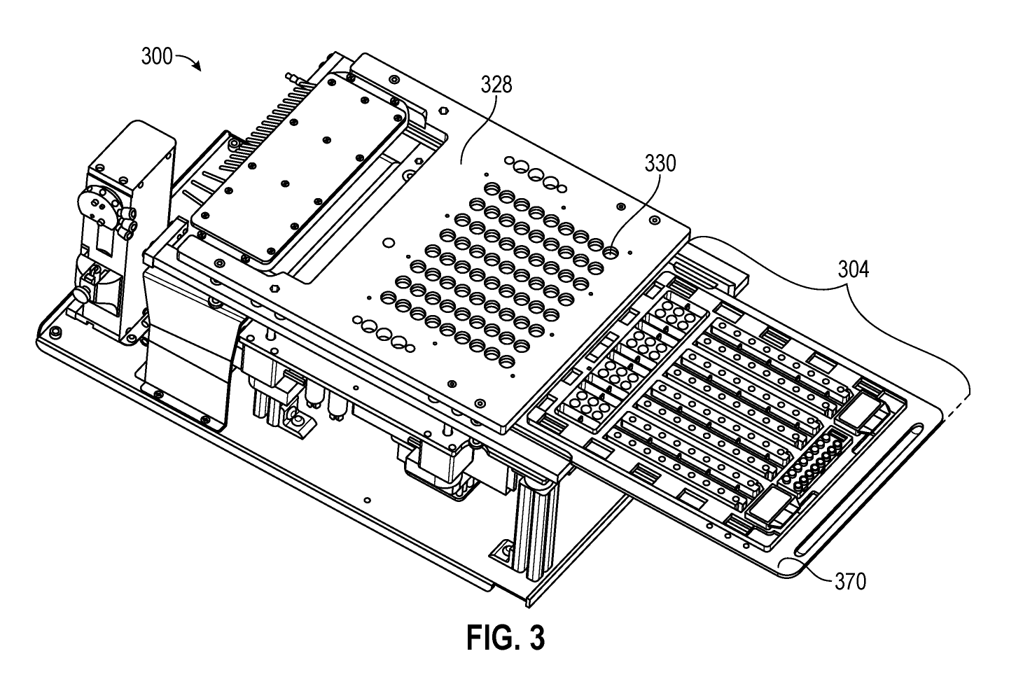

In some embodiments, the one or more cartridge bays can process nucleic acid preparation cartridges, in any combination. In some embodiments, each cartridge bay is loaded, e.g., by the operator or by a robotic assembly. FIG. 3 depicts an exemplary drawing of a microfluidics cartridge bay assembly 300. In some embodiments, a cartridge is loaded into a bay when the bay is in the open position by placing the cartridge into a carrier plate 370 to form a carrier plate assembly 304. The carrier plate is itself, in some embodiments, a stand-alone component which may be removed from the cartridge bay. This cartridge bay holds the cartridge in a known position relative to the instrument. In some embodiments, a lid 328 (e.g., a heated lid) comprises one or more holes 330 to facilitate the processing and/or monitoring of reactions occurring in one or more vessels. In some embodiments, prior to loading a new cartridge onto the instrument, a primer cassette may be installed onto the cartridge. In some embodiments, the primer cassette would be packaged separately from the cartridge. In some embodiments, a primer cassette may be placed into a cartridge. In some embodiments, both primer cassettes and cartridges would be identified such that placing them onto the instrument allows the instrument to read them (e.g., using a barcode scanner) and initiate a protocol associated with the cassettes.

In some embodiments, prior to installing a carrier into the instrument, bulk reagents may be loaded into the carrier. In some embodiments, a user or robotic assembly may be informed as to which reagents to load and where to load them by the instrument or an interface on a remote sample loading station. In some embodiments, after loading a cartridge with a primer cassette into an instrument, a user would have the option of choosing certain reaction conditions (e.g., a number of PCR cycles) and/or the quantity of samples to be run on the cartridge. In some embodiments, each cartridge may have a capacity of 1, 2, 3, 4, 5, 6, 7, 8, 9, 10 or more samples.

In some embodiments, systems provided herein may be configured to process RNA. However, in some embodiments, the system may be configured to process DNA. In some embodiments, different nucleic acids may be processed in series or in parallel within the system. In some embodiments, cartridges may be used to perform gene fusion assays in an automated fashion, for example, to detect genetic alterations in ALK, RET, or ROS1. Such assays are disclosed herein as well as in US Patent Application Publication Number US 2013/0303461, which was published on Nov. 14, 2013, US Patent Application Publication Number and US 2015/02011050, which was published on Jul. 20, 2013, the contents of each of which are incorporated herein by reference in their entirety. In some embodiments, systems provided herein can process in an automated fashion an Xgen protocol from Integrated DNA Technologies or other similar nucleic acid processing protocol.

In some embodiments, cartridge and cassettes will have all of the reagents needed for carrying out a particular protocol. In some embodiments, once a carrier is loaded into a cartridge bay an access door to that bay is closed, and optionally a lid (e.g., a heated lid) may be lowered automatically. In some embodiments, lowering of the lid (e.g., the heated lid) forces (or places) the cartridge down onto an array of heater jackets which conform to each of a set of one or more temperature controlled vessels in the cartridge. In some embodiments, this places the cartridge in a known position vertically in the drawer assembly. In some embodiments, lowering of the lid forces the cartridge down into a position in which rotary valves present in the cartridge are capable of engaging with corresponding drivers that control the rotational position of the valves in the cartridge. In some embodiments, automation components are provided to ensure that the rotary valves properly engage with their drivers.

In some embodiments of methods provided herein, a nucleic acid sample present in a cartridge (e.g., within a vessel of a cassette) will be mixed with a lyosphere. In some embodiments, the lyosphere will contain a fluorophore which will attach to the sample. In some embodiments, there will also be a “reference material” in the lyosphere which will contain a known amount of a molecule (e.g., of synthetic DNA). In some embodiments, attached to the “reference material” will be another fluorophore which will emit light at a different wavelength than the sample's fluorophore. In some embodiments, fluorophores used may be attached to the sample or the “reference material” via an intercalating dye (e.g., SYBR Green) or a reporter/quencher chemistry (e.g., TaqMan, etc.). In some embodiments, during quantitative PCR (qPCR) cycling the fluorescence of the two fluorophores will be monitored and then used to determine the amount of nucleic acid (e.g., DNA, cDNA) in the sample by the Comparative CT method.

Advantageously, certain systems described herein may include modular components (e.g., cassettes) that can allow tailoring of specific reactions and/or steps to be performed. In some embodiments, certain cassettes for performing a particular type of reaction are included in the cartridge. For example, cassettes including vessels containing lyospheres with different reagents for performing multiple steps of a PCR reaction may be present in the cartridge. The frame or cartridge may further include empty regions for a user to insert one or more cassettes containing specific fluids and/or reagents for a specific reaction (or set of reactions) to be performed in the cartridge. For example, a user may insert one or more cassettes containing particular buffers, reagents, alcohols, and/or primers into the frame or cartridge. Alternatively, a user may insert a different set of cassettes including a different set of fluids and/or reagents into the empty regions of the frame or cassette for performing a different reaction and/or experiment. After the cassettes are inserted into the frame or cartridge, they may form a fluidic connection with a channel system for transporting fluids to conduct the reactions/analyses.

In some embodiments, multiple analyses may be performed simultaneously or sequentially by inserting different cassettes into the cartridge. For instance, the systems and methods described herein may advantageously provide the ability to analyze two or more samples without the need to open the system or change the cartridge. For example, in some cases, one or more reactions with one or more samples may be conducted in parallel (e.g., conducting two or more PCR reactions in parallel). Such modularity and flexibility may allow for the analysis of multiple samples, each of which may require one or several reaction steps within a single fluidic system. Accordingly, multiple complex reactions and analyses may be performed using the systems and methods described herein.

Unlike certain existing fluidic systems and methods, the systems and methods described herein may be reusable (e.g., a reusable carrier plate) or disposable (e.g., consumable components including cassettes and various fluidic components). In some cases, the systems described herein may occupy a relatively small footprint as compared to certain existing fluidic systems for performing similar reactions and experiments.

In some embodiments, the cassettes and/or cartridge includes stored fluids and/or reagents needed to perform a particular reaction or analysis (or set of reactions or analyses) with one or more samples. Examples of cassettes include, but are not limited to, reagent cassettes, primer cassettes, buffer cassettes, waste cassettes, sample cassettes, and output cassettes. Other appropriate modules or cassettes may be used. Such cassettes may be configured in a manner that prevents or eliminates contamination or loss of the stored reagents prior to the use of those reagents. Other advantages are described in more detail below.

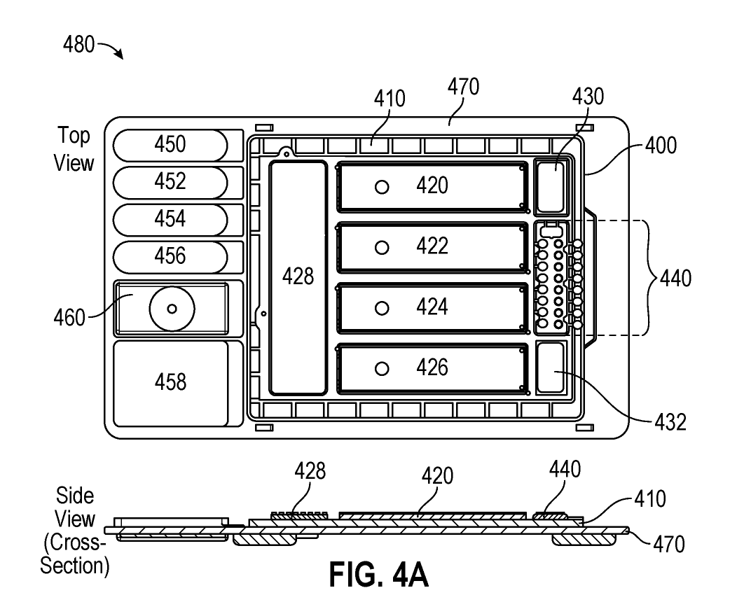

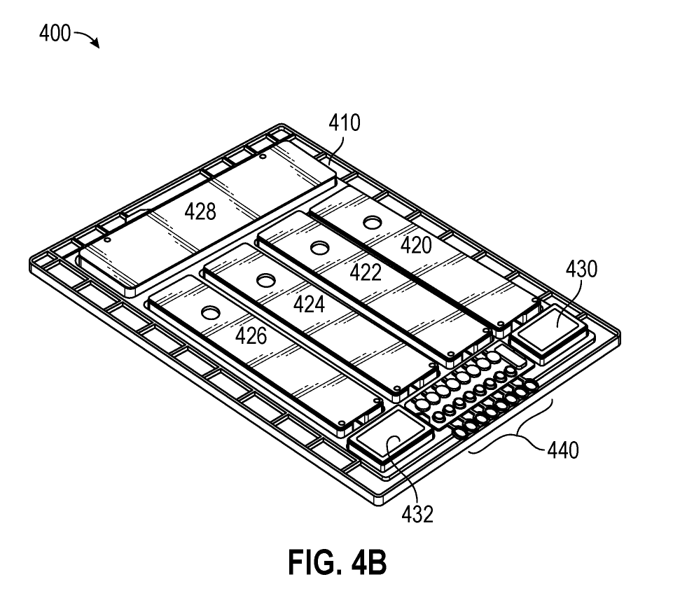

In one embodiment, as shown illustratively in FIGS. 4A and 4B, cartridge 400 comprises a frame 410 and cassettes 420, 422, 424, 426, 428, 430, 432, and 440. In some embodiments, each of these cassettes may be in fluidic communication with a channel system (e.g., positioned underneath the cassettes, not shown). In some embodiments, at least one of cassettes 428 (e.g., a reagent cassettes), 430 (e.g., a reagent cassette), and 432 (e.g., a reagent cassette) may be inserted into frame 410 by the user such that the cassettes are in fluidic communication with the channel system. For example, in some embodiments, one of cassettes 428, 430, and 432 is a reagent cassette containing a reaction buffer (e.g., Tris buffer). In certain embodiments, cassettes 428, 430 and/or 432 may comprise one or more reagents and/or reaction vessels for a reaction or a set of reactions. In some embodiments, module 440 comprises a plurality of sample wells and/or output wells (e.g., samples wells configured to receive one or more samples). In some cases, cassettes 420, 422, 424, and 426 may comprise one or more stored reagents or reactants (e.g., lyospheres). For instance, each of cassettes 420, 422, 424, and 426 may include different sets of stored reagents or reactants for performing separate reactions. For example, cassette 420 may include a first set of reagents for performing a first PCR reaction, and cassette 422 may include a second set of reagents for performing a second PCR reaction. The first and second reactions may be performed simultaneously (e.g., in parallel) or sequentially.

In some embodiments, as shown illustratively in FIG. 4A, a carrier plate assembly 480 comprises a carrier plate 470 and additional cassettes including modules 450, 452, 454, 456, 458, and 460. In an exemplary embodiment, cassettes 450, 452, 454, 456, 458, and 460 may each comprise one or more stored reagents and/or may be configured and arranged to receive one or more fluids (e.g., module 458 may be a waste module configured to collect reaction waste fluids). In some embodiments, one or more of cassettes 450, 452, 454, 456, 458, and 460 may be refillable.

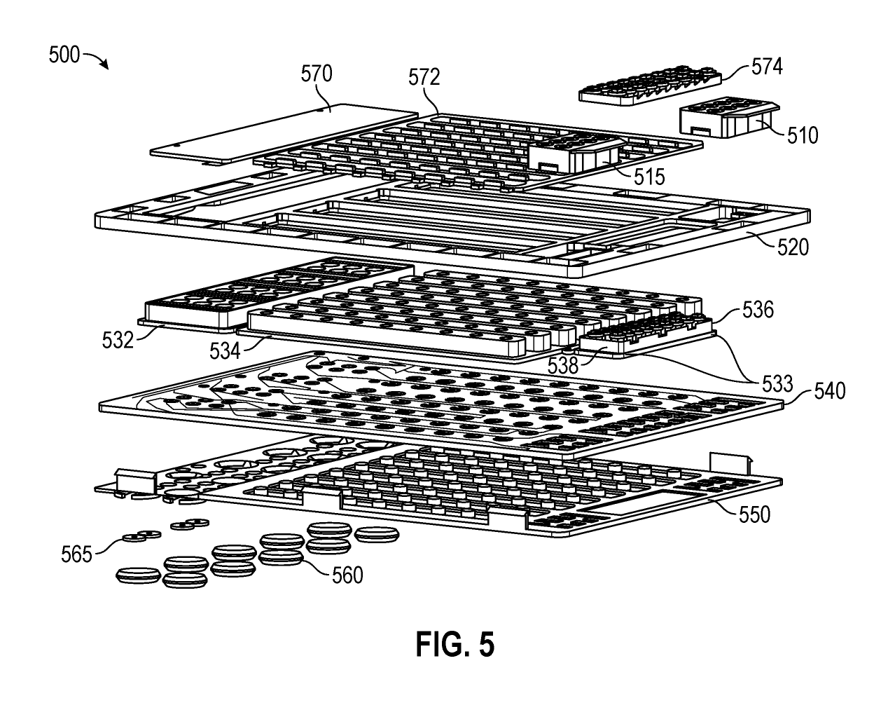

FIG. 5 is an exploded view of an exemplary cartridge 500, according to one set of embodiments. Cartridge 500 comprises a primer cassette 510 and a primer cassette 515 which may be inserted into one or more openings in a frame 520. Cartridge 500 further comprises a fluidics layer assembly 540 containing a channel system adjacent and non-integral to frame 520. In some embodiments, a set of cassettes 532 (e.g., comprising one or more primer cassettes, buffer cassettes, reagent cassettes, and/or waste cassettes, each optionally including one or more vessels), set of reaction cassettes 534, which comprises reaction vessels, an input/output cassette 533, which comprises sample input vessels 536 and output vessels 538, may be inserted into one or more openings in frame 520. In some embodiments, cartridge 500 comprises a valve plate 550. In some embodiments, valve plate 550 connects (e.g., snaps) into frame 520 and holds in place fluidics layer assembly 540 and cassettes 532, 533 and 534 in frame 520. In certain embodiments, cartridge 500 comprises valves 560, as described herein, and a plurality of seals 565. In some cases, frame 520 and/or one or more modules may be covered by covers 570, 572, and/or 574.

### Microfluidic System and Methods

As noted above, fluidic systems including cartridges with modular components (cassettes) and/or microfluidic channels for performing chemical and/or biological analyses are provided. The systems described herein include a cartridge comprising, in some embodiments, a frame, one or more cassettes which may be inserted into the frame, and a channel system for transporting fluids. In certain embodiments, the one or more cassettes comprise one or more reservoirs or vessels configured to contain and/or receive a fluid (e.g., a stored reagent, a sample). In some cases, the stored reagent may include one or more lyospheres. The systems and methods described herein may be useful for performing chemical and/or biological reactions including polymerase chain reactions (PCR) such as those performed within a laboratory, clinical (e.g., hospital), or research setting.

Advantageously, certain systems described herein may include modular components (e.g., cassettes) that can allow tailoring of specific reactions and/or steps to be performed. In some embodiments, certain cassettes for performing a particular type of reaction are included in the cartridge by a manufacturer. For example, cassettes including vessels containing lyospheres with different reagents for performing multiple steps of a PCR reaction may be present in the cartridge. The frame or cartridge may further include empty regions for a user to insert one or more cassettes containing specific fluids and/or reagents for a specific reaction (or set of reactions) to be performed in the cartridge. For example, a user may insert one or more cassettes containing particular buffers, reagents, alcohols, and/or primers into the frame or cartridge. Alternatively, a user may insert a different set of cassettes including a different set of fluids and/or reagents into the empty regions of the frame or cassette for performing a different reaction and/or experiment. After the cassettes are inserted into the frame or cartridge, they may form a fluidic connection with a channel system for transporting fluids to conduct the reactions/analyses.

In some embodiments, multiple analyses may be performed simultaneously or sequentially by inserting different cassettes into the cartridge. For instance, the systems and methods described herein may advantageously provide the ability to analyze two or more samples without the need to open the system or change the cartridge. For example, in some cases, one or more reactions with one or more samples may be conducted in parallel (e.g., conducting two or more PCR reactions in parallel). Such modularity and flexibility may allow for the analysis of multiple samples, each of which may require one or several reaction steps within a single fluidic system. Accordingly, multiple complex reactions and analyses may be performed using the systems and methods described herein.

Unlike certain existing fluidic systems and methods, the systems and methods described herein may be reusable (e.g., a reusable carrier plate) or disposable (e.g., consumable components including cassettes and various fluidic components). In some cases, the systems described herein may occupy a relatively small footprint as compared to certain existing fluidic systems for performing similar reactions and experiments.

In some embodiments, the cassettes and/or cartridge includes stored fluids and/or reagents needed to perform a particular reaction or analysis (or set of reactions or analyses) with one or more samples. Examples of cassettes include, but are not limited to, reagent cassettes, primer cassettes, buffer cassettes, waste cassettes, sample cassettes, and output cassettes. Such cassettes may be configured in a manner that prevents or eliminates contamination or loss of the stored reagents prior to the use of those reagents. Other advantages are described in more detail herein.

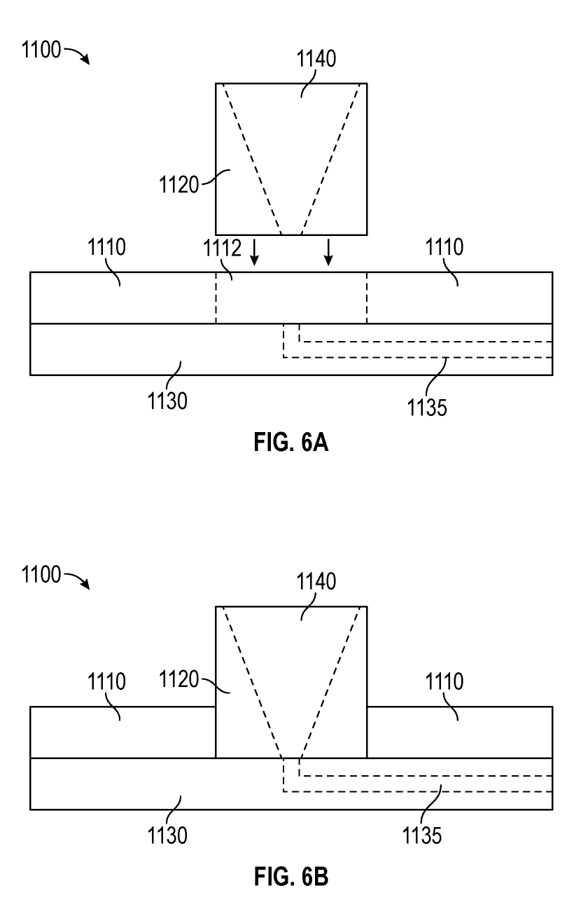

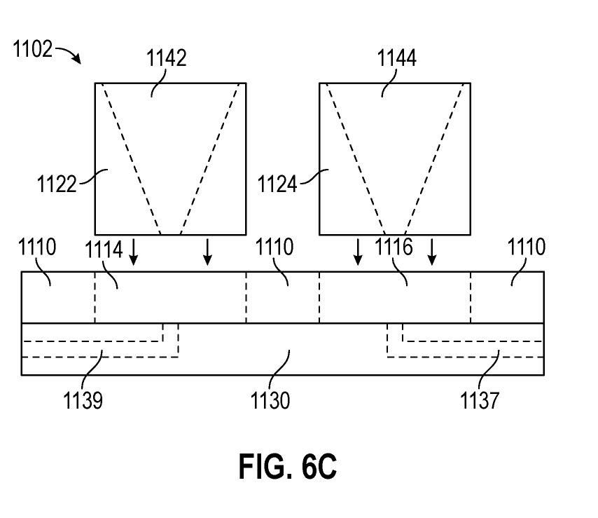

As described herein, a cartridge may include a frame or support structure for inserting one or more cassettes and a channel system that can be fluidically connected to the fluidic components (e.g., vessels, reservoirs) within a cassette. In some embodiments, the channel system is adjacent to and non-integral to the frame. That is, in certain embodiments, the frame does not include the channels of the channel system formed therein. In some such embodiments, the channel system is formed independently from the frame and is located adjacent (e.g., directly adjacent) the frame. For example, as shown illustratively in FIG. 6A, cartridge 1100 comprises frame 1110 and channel system 1130 located adjacent and non-integral to frame 1110. In some embodiments, channel system 1130 comprises at least one fluidic (e.g., microfluidic) channel 1135. Channel systems and fluidic channels are described in more detail below. In some embodiments, the frame and/or cartridge is in direct contact with a carrier plate. The carrier plate may be configured, in certain embodiments, to facilitate transport of the cartridge and/or proper insertion of the cartridge into an analysis device or instrument.

In some embodiments, the frame comprises at least one opening. For example, referring again to FIG. 6A, frame 1110 includes opening 1112. The opening may be constructed and arranged to house a cassette. For example, in some embodiments, cartridge 1120 may be inserted into (e.g., positioned into) opening 1112. In some embodiments, cartridge 1120 includes vessel 1140. As described in more detail below, a module may include one or more fluids and/or reagents for a particular reaction or analysis, or may be configured to receive one or more fluids and/or reagents for a particular reaction or analysis (e.g., a waste cartridge, a reagent cartridge, a primer cartridge, a buffer cartridge, a sample cartridges, an output cartridge). In some cases, the one or more fluids and/or reagents may be present in one or more vessels of the cartridge (e.g., during storage, during use). The cartridge may be inserted into the cartridge or frame by a user, in some cases. The cartridge may be configured such that the opening of its frame allows fluidic communication between the cartridge and the channel system (e.g., a channel, port, or other fluidic component of the channel system).

Referring now to FIG. 6B, cartridge 1100 comprises frame 1110, channel system 1130, and cartridge 1120 inserted into frame 1110 such that cartridge 1120 is adjacent to channel system 1130. In some embodiments, cartridge 1120 is in fluidic communication with channel system 1130, such as fluidic channel 1135 (e.g., vessel 1140 of cartridge 1120 may be in fluidic communication with fluidic channel 1135).

For example, in some embodiments, the cartridge may already be present in the cartridge upon use of the cartridge (e.g., performing of a reaction within the cartridge) by the user. In certain embodiments, the cartridge may be present in the cartridge upon and/or during fabrication of the cartridge (e.g., the cartridge may be inserted into the frame/cartridge by the manufacturer). In some cases, the cartridge may be physically connected to the cartridge. For example, in some such embodiments, the cartridge may be connected to, and configured to maintain contact with, a surface of the channel system via an adhesive (e.g., an epoxy), a mechanical mechanism (e.g., a groove, a latch), friction, or by other means known in the art. Connection of cartridge module to the cartridge may be conducted by the manufacturer and/or by the user, in some cases.

In certain embodiments, a frame comprises two or more openings each configured to receive a cartridge. Referring now to FIG. 6C, a cartridge 1102 comprises frame 1110 adjacent channel system 1130. In some embodiments, frame 1110 comprises two or more openings such as a first opening 1114 and a second opening 1116. In certain embodiments, a first cassette may be inserted into the first opening and a second cassette may be inserted into the second opening. For instance, as shown illustratively, first cassette 1122 may be inserted into first opening 1114 and/or second cassette 1124 may be inserted into second opening 1124.

In some embodiments, cassette 1122 and/or cassette 1124 contains, or is configured to contain, one or more fluids (e.g., a buffer) and/or reagents. For example, in certain embodiments, the fluid(s) and/or reagent(s) may be contained within vessel 1142 and/or 1144. Additional vessels, optionally containing one or more fluids and/or reagents, may also be present in the cassette (not shown). In some embodiments, the fluid(s) and/or reagent(s) are introduced into the cartridge and/or channel system after the cassettes are inserted into opening 1114 and/or 1116. For example, as described herein, a fluid and/or reagent present in the cassette may be transported to the channel system (e.g., via a fluidic channel in the channel system).

In another embodiment, the fluid(s) and/or the reagent(s) are introduced into a fluidic component of the cassette. For instance, in some cases, one or more fluids and/or reagents may be transported from the channels system to the cassette. For example, a fluid and/or reagent in the channel system may be transported to a first cassette (e.g., a fluid and/or reagent in fluidic channel 1139/1137 may be transferred to cassette 1122/1124 inserted into the cartridge) where a first reaction can take place. In one particular set of embodiments, the resulting fluid may be transported back to the channel system (e.g., from cassette 1122/1124 into fluidic channel 1139/1137). In certain embodiments, the resulting fluid may be transported to a second cassette (e.g., in vessel 1144 of cassette 1124) where a second reaction can take place. In some embodiments, the cartridge may comprise a plurality of openings and/or cassettes configured and arranged such that a plurality of reactions make take place within/amongst the cassettes.

In some embodiments, the frame comprises at least 2, at least 3, at least 4, at least 5, at least 6, at least 8, at least 10, at least 12, at least 16, or at least 20 openings configured to receive one or more cassettes. In certain embodiments, the frame comprises less than or equal to 24, less than or equal to 20, less than or equal to 16, less than or equal to 12, less than or equal to 10, less than or equal to 8, less than or equal to 6, less than or equal to 5, less than or equal to 4, or less than or equal to 3 openings configured to receive one or more modules. Combinations of the above-referenced ranges are also possible (e.g., at least 2 and less than or equal to 24 openings). Other ranges are also possible. In some such embodiments, the cartridge is configured to allow fluidic communication between the first cassette and the channel system (e.g., at least one channel of the channel system) and/or the second cassette and the channel system (e.g., the at least one channel of the channel system) upon insertion of the first and/or second cassette into the frame, respectively.

In some embodiments, the cartridge is configured to allow fluidic communication between a third cassette and/or a fourth cassette and the channel system. Additional cassettes are also possible and are described in more detail below.

As described herein, in some embodiments, one or more cassettes may include reagents for a particular reaction or analysis. For example, one or more cassettes inserted and/or fixed to the cartridge and/or the frame may contain one or a series of stored reagents therein, e.g., for performing a particular reaction or analysis on the cartridge. In certain embodiments, one or more cassettes may include fluids (e.g., a buffer) for a particular reaction or analysis. In some cases, the cassette may include an area for a particular reaction to take place (e.g., a vessel). As such, different configurations of cassettes may be used to tailor specific reactions and/or processes to be performed with the cartridge.

As described herein, the cartridge is configured, in some embodiments, to allow fluidic communication between at least one cassette and the channel system (e.g., a channel of the channel system). In some embodiments, one or more cassette are inserted into or arranged within a cartridge and/or a frame such that at least one cassette is in fluidic communication with the channel system. In certain embodiments at least one of the cassettes (or fluidic component(s) of the cassettes) is/are not in fluid communication with the channel system prior to insertion of the cassette into the cartridge (e.g., into an opening of the frame), but fluid communication between the module and the channel system may occur upon or after insertion of the cassette into the cartridge. For example, referring again to FIG. 6B, cassette 1120, upon or after insertion into frame 1110 may be in fluidic communication with channel system 1130.

In some cases, one or more cassettes may contain a reagent (e.g., a stored reagent) therein. For example, the cassette may include a fluidic component such as a reservoir, vessel, and/or a channel (e.g., a microfluidic channel) that can contain one or more reagents (e.g., stored reagents) therein. Two or more cassettes may contain different reagents depending on the desired reaction (e.g., a first reagent for conducting a first reaction and a second reagent for conducting a second reaction). For example, in some embodiments, a first cassette is constructed and arranged for conducting a first reaction (e.g., a first PCR reaction) and a second cassette is constructed and arranged for conducting a second reaction (e.g., a second PCR reaction) independent of the first reaction.

In some cases, the one or more cassettes may be sealed. For example, in some embodiments, the cassettes may contain a fluid such that, prior to insertion of the cassette into the frame, the fluid does not substantially escape (e.g., by leaking, by evaporation) the cassette. Advantageously, sealing the cassettes may reduce or prevent evaporation of a stored liquid reagent and/or contamination of reagents. In some embodiments, the frame or cartridge includes one or more puncture components constructed and arranged to puncture one or more portions of the cassette upon insertion of the cassette into the frame or cartridge. In some such embodiments, upon insertion of the cassette into an opening of the frame, the puncturing component (e.g., located within or adjacent the opening into which the cassette is being inserted) punctures the cassette such that a reagent contained within the cassette is in fluidic communication with the channel system.

In some embodiments, at least one of the cassettes contains two or more reagents stored therein, not in fluid communication with one another prior to insertion of the module into the cartridge. For example, in some embodiments, at least one cassette comprises a first reagent stored therein and a second reagent stored therein, wherein the first and second reagents are not in fluid communication with one another prior to insertion of the cassette into the cartridge. In certain embodiments, at least one of the cassettes is inserted or fixed in the cartridge such that a first reagent and/or a second reagent stored therein are in fluid communication with the channel system (e.g., at least one channel of the channel system).

In some embodiments, at least one of the cassettes has a total working volume of at least 0.1 mL and/or less than or equal to 25 mL. In certain embodiments, at least one of the cassettes has a total working volume of at least 0.1 mL, at least 0.2 mL, at least 0.5 mL, at least about 1 mL, at least about 2 mL, at least about 5 mL, at least about 10 mL, at least about 15 mL, or least about 20 mL. In some embodiments, at least one of the cassettes has a total working volume of less than or equal to 25 mL, less than or equal to 20 mL, less than or equal to 15 mL, less than or equal to 10 mL, less than or equal to 5 mL, less than or equal to 2 mL, less than or equal to 1 mL, less than or equal to 0.5 mL, less than or equal to 0.2 mL. Combinations of the above referenced ranges are also possible (e.g., at least 0.1 mL and less than or equal to 25 mL). Other ranges are also possible.

In some cases, at least one of the modules may be refillable. For example, at least one module may be used to perform a reaction (e.g., between a stored reagent therein and a sample) and the module may be refilled with a new reagent after the first reaction is completed. In some such embodiments, the at least one module may be used to perform two or more reactions. In other embodiments, a module is non-refillable.

In some embodiments, one or more modules comprise one or more sample wells (i.e., a sample module). For example, in certain embodiments, a module comprising one or more sample wells may be fixed to the cartridge and in fluidic communication with the channel system. In some such embodiments, a user may insert (e.g., via pipetting) one or more samples into the one or more sample wells. The sample(s) may be transported into the channel system and into one or more reservoirs or vessels for conducting a reaction or analysis. In some embodiments, each module comprising one or more sample wells has a total volume of at least about 5 μL (e.g., at least about 10 μL, at least about 20 μL, at least about 30 μL, at least about 40 μL, at least about 50 μL, at least about 80 μL, at least about 100 μL, at least about 200 μL) and/or less than or equal to 500 μL (e.g., less than or equal to 400 μL, less than or equal to 300 μL, less than or equal to 200 μL, less than or equal to 100 μL, less than or equal to 80 μL, less than or equal to 60 μL, less than or equal to 40 μL, less than or equal to 20 μL). Combinations of the above-referenced ranges are also possible.

In certain embodiments, one or more modules comprise one or more output wells (i.e., an output module). For example, in some cases, the cartridge may be configured and arranged such that one or more samples react with one or more reagents present (or introduced) in the cartridge, and the product(s) of the reaction(s) is/are transferred to the output wells. In some embodiments, each module comprising one or more output wells has a total volume of at least about 5 μL (e.g., at least about 10 μL, at least about 20 μL, at least about 30 μL, at least about 40 μL, at least about 50 μL, at least about 80 μL, at least about 100 μL, at least about 200 μL) and/or less than or equal to 500 μL (e.g., less than or equal to 400 μL, less than or equal to 300 μL, less than or equal to 200 μL, less than or equal to 100 μL, less than or equal to 80 μL, less than or equal to 60 μL, less than or equal to 40 μL, less than or equal to 20 μL). Combinations of the above-referenced ranges are also possible.

In some embodiments, one or more modules comprise one or more waste modules. For example, in some embodiments, byproducts and/or unused reagent may be transferred from the channel system during operation to the one or more waste modules. In some embodiments, the waste module has a volume of at least 0.1 mL and/or less than or equal to 5 mL. In certain embodiments the waste module has a volume of at least 0.1 mL, at least 0.2 mL, at least 0.5 mL, at least about 1 mL, at least about 2 mL, at least about 3 mL, at least about 4 mL. In some embodiments, the waste module has a volume of less than or equal to 5 mL, less than or equal to 2 mL, less than or equal to 1 mL, less than or equal to 0.5 mL, less than or equal to 0.2 mL. Combinations of the above referenced ranges are also possible (e.g., at least 0.1 mL and less than or equal to 3 mL). Other ranges are also possible.

In some cases, one or more modules are configured to receive a fluid such that a reaction may take place within the module(s). For example, in some embodiments, one or more modules may receive a reactant and a sample such that the reactant and sample react within the one or more modules. In some embodiments, one or more modules comprise a reagent (i.e., a reagent module), a primer (i.e., a primer module), or a buffer (i.e., a buffer module). For example, in some cases, the reactant module contains one or more lyospheres, as described in more detail below. Reactants, reagents, primers, and buffers are also described in more detail below.

In certain embodiments, a cartridge may comprise a combination of different types of cassettes. Some modules may be inserted by the user and other modules may be fixed to the cartridge (e.g., fixed to the frame). In some embodiments, a cartridge comprises 1 or more, 2 or more, 3 or more, 4 or more, 5 or more, 6 or more, 8 or more, 10 or more, 12 or more, 16 or more, or 20 or more modules and/or openings configured to receive a module. In some cases, the cartridge may comprise a combination of one or more sample modules, one or more modules output modules, one or more waste modules, one or more insertable modules, and/or one or more fixed modules containing a stored reagent.

As noted above, in one embodiment, as shown illustratively in FIGS. 4A and 4B, cartridge 400 comprises a frame 410 and cassettes 420, 422, 424, 426, 428, 430, 432, and 440. In some embodiments, each of these cassettes may be in fluidic communication with a channel system (e.g., positioned underneath the cassettes, not shown). In some embodiments, at least one of cassettes 428 (e.g., a reagent cassettes), 430 (e.g., a reagent cassette), and 432 (e.g., a reagent cassette) may be inserted into frame 410 by the user such that the cassettes are in fluidic communication with the channel system. For example, in some embodiments, one of cassettes 428, 430, and 432 is a reagent cassette containing a reaction buffer (e.g., Tris buffer). In certain embodiments, cassettes 428, 430 and/or 432 may comprise one or more reagents and/or reaction vessels for a reaction or a set of reactions. In some embodiments, module 440 comprises a plurality of sample wells and/or output wells (e.g., samples wells configured to receive one or more samples). In some cases, cassettes 420, 422, 424, and 426 may comprise one or more stored reagents or reactants (e.g., lyospheres). For instance, each of cassettes 420, 422, 424, and 426 may include different sets of stored reagents or reactants for performing separate reactions. For example, cassette 420 may include a first set of reagents for performing a first PCR reaction, and cassette 422 may include a second set of reagents for performing a second PCR reaction. The first and second reactions may be performed simultaneously (e.g., in parallel) or sequentially.

In some embodiments, as shown illustratively in FIG. 4A, a carrier plate assembly 480 comprises a carrier plate 470 and additional cassettes including modules 450, 452, 454, 456, 458, and 460. In an exemplary embodiment, cassettes 450, 452, 454, 456, 458, and 460 may each comprise one or more stored reagents and/or may be configured and arranged to receive one or more fluids (e.g., module 458 may be a waste module configured to collect reaction waste fluids). In some embodiments, one or more of cassettes 450, 452, 454, 456, 458, and 460 may be refillable.

In some embodiments, at least one cassette comprises one or more reservoirs. In some cases, the reservoir is a vessel that can be used to contain a fluid and/or reagent. While much of the description herein relates to vessels, those skilled in the art would understand based upon the teaching of this specification that other types of reservoirs are also possible including, but not limited to, conduits, channels, microchannels, cavities, capsules, pits, pores, and the like.

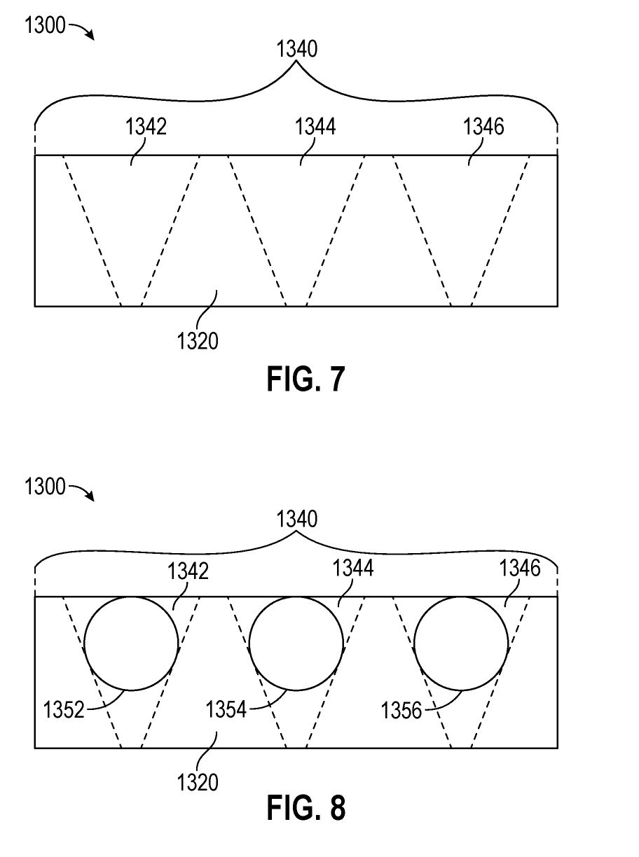

In certain embodiments, at least one cassette comprises a set of vessels. As shown illustratively in FIG. 7, cassette 1320 includes a set of vessels 1340 including vessels 1342, 1344, and 1346. A cartridge or carrier plate may include one or more such cassettes in some instances. For example, in some embodiments, a first cassette comprises a first set of vessels and a second cassette comprises a second set of vessels. In certain embodiments, a third cassette may comprise a third set of vessels. Additional sets of vessels are also possible. In some embodiments, at least one set of vessels includes at least 2, at least 4, at least 6, at least 8, at least 10, or at least 15 vessels. In certain embodiments, at least one set of vessels includes less than or equal to 20 vessels, less than or equal to 15 vessels, less than or equal to 10 vessels, less than or equal to 8 vessels, less than or equal to 6 vessels, or less than or equal to 4 vessels. Combinations of the above-referenced ranges are also possible (e.g., at least 2 and less than or equal to 4 vessels). Other ranges are also possible.

As described herein, a cartridge is configured, in some embodiments, to allow fluidic communication between at least one vessel and the channel system (e.g., a channel of the channel system). Referring again to FIG. 6B, cartridge 1100 comprises frame 1110, channel system 1130 including fluidic channel 1135, and cassette 1120 including vessel 1140 positioned in frame 1110 such that vessel 1140 is in fluidic communication with fluidic channel 1135. In some embodiments, fluidic communication occurs (e.g., substantially simultaneously) between each of the vessels in a module and the channel system (e.g., each vessel in a module may be in fluidic communication with a vessel channel of the channel system).

In some cases, at least one vessel may contain a reagent (e.g., a stored reagent) therein. Two or more vessels may contain different reagents depending on the desired reaction (e.g., a first reagent for conducting a first reaction and a second reagent for conducting a second reaction). For example, in some embodiments, a first vessel is constructed and arranged for conducting a first reaction (e.g., a first part of a PCR reaction) and a second vessel is constructed and arranged for conducting a second reaction (e.g., a second part of a PCR reaction) independent of the first reaction. The two or more vessels may be in fluidic communication with the channel system such that fluids can be transported between each of the vessels. For example, after a first reaction (e.g., between a sample component and a first reagent positioned within the first vessel) takes place in a first vessel, the reaction product may be transported from the first vessel back tithe channel system and into the second vessel for conducting a second reaction (e.g., between the reaction product and a second reagent). The same process can be used to transport fluids into various vessels for conducting various reactions (e.g., sequential reactions).

In some cases, the one or more vessels may be sealed (e.g., using a suitable foil, membrane, cover, etc.). For example, in some embodiments, the vessel may contain a fluid such that, prior to insertion of the cassette into the cartridge, the fluid does not substantially escape the vessel (e.g., by leaking, by evaporation). Advantageously, sealing the vessels may reduce or prevent evaporation of a stored liquid reagent.

In some embodiments, the cartridge (or frame) comprises one or more puncture components constructed and arranged to puncture one or more portions of the vessel upon insertion of the vessel into the frame. In some such embodiments, upon insertion of the vessel into an opening of the cartridge or frame, the puncturing component (e.g., located within or adjacent the opening into which the cassette comprising the vessel is being inserted) punctures the vessel such that the vessel and/or a reagent contained within the vessel is in fluidic communication with the channel system. In some embodiments, a cartridge includes a series of puncture components, each puncture component aligned to puncture a seal of a vessel. A puncture component may be in any suitable form, such as, for example, a probe with a beveled leading edge.

In certain embodiments, the vessels in the set of vessels (e.g., of a cassette) are not in fluid communication with each other prior their insertion into the cartridge (e.g., prior to insertion of the cassette comprising the set of vessels into an opening of the cartridge and/or the frame). In some embodiments, a set of vessels contains two or more reagents stored therein, not in fluid communication with one another prior to insertion of the cassette including the vessels into the cartridge. For example, in some embodiments, a set of vessels comprises a first reagent stored in a first vessel therein and a second reagent stored in a second vessel therein, wherein the first and second reagents (and/or the first and second vessels) are not in fluid communication with one another prior to insertion of the cassette including the set of vessels into the cartridge. In certain embodiments, the set of vessels is inserted or fixed in the cartridge such that a first reagent and/or a second reagent stored therein are in fluid communication with the channel system (e.g., at least one channel of the channel system).

In some embodiments, at least one vessel may be configured to receive a fluid (e.g., a fluid containing a sample, a reaction fluid, a waste fluid, etc.), such as to receive a fluid from the channel system. In certain embodiments, a reaction may be performed in a vessel. For example, the vessel may contain a first reagent (e.g., a first stored reagent) and the first reagent is reacted with a fluid to form a second fluid. In some cases, the reaction may be a chemical and/or biological reaction.

In some embodiments, at least one vessel may be configured to deliver a fluid (e.g., a fluid containing a sample, a reaction fluid, etc.), such as to deliver a fluid to the channel system. In some cases, at least one of the vessels may be refillable. For example, at least one vessel may be used to perform a reaction (e.g., between a stored reagent therein and a sample) and the vessel may be refilled with a new reagent after the first reaction is completed. In some such embodiments, the at least one vessel may be used to perform two or more reactions.

In certain embodiments, at least one of the vessels has a volume of at least about 1 μL (e.g., volume of at least about 5 μL, at least about 10 μL, at least about 20 μL, at least about 30 μL, at least about 40 μL, at least about 50 μL, at least about 80 μL, at least about 100 μL, at least about 200 μL) and/or less than or equal to 500 μL (e.g., less than or equal to 400 μL, less than or equal to 300 μL, less than or equal to 200 μL, less than or equal to 100 μL, less than or equal to 80 μL, less than or equal to 60 μL, less than or equal to 40 μL, less than or equal to 20 μL). Combinations of the above-referenced ranges are also possible. The volume may be defined by the volume encompassed by the vessel sidewalls and a vessel cover (if present).

A vessel may have any suitable shape. In some embodiments, at least one vessel has a conical shape. In certain embodiments, at least one vessel has a tapered cross-sectional shape. As described herein, the vessel may have any suitable shape. In some embodiments, at least one vessel has a conical shape. In certain embodiments, at least one vessel has a tapered cross-sectional shape defined by the sidewall(s). The tapered shape may be substantially conical, like that of vessel 1140 shown in FIG. 6A, or may include some degree of curvature (e.g., the sidewall(s) may be/appear curved rather than straight from the perspective shown in FIG. 6A). The apex of the conical or tapered shape may be approximated as the inlet to the vessel. The vessel may have a taper angle defined as the angle formed between the axis and the surface (e.g., sidewall). In some embodiments the taper angle of the vessel may be at least 50, at least 10°, at least 20°, at least 30°, at least 40°, at least 45°, at least 50°, at least 60°, or at least 70°. In some embodiments the taper angle of the vessel may be less than or equal to 80°, less than or equal to 70°, less than or equal to 60°, less than or equal to 50°, less than or equal to 45°, less than or equal to 40°, less than or equal to 30°, less than or equal to 20°, or less than or equal to 10°. Combinations of the above-referenced ranges are also possible (e.g., at least 20° and less than or equal to 45°). Other ranges are also possible.

The shape of the vessel may be such that the vessel is free of a ledge (i.e., ledge free); such a configuration may facilitate mixing and/or reduce the presence of residue in the vessel. The shape of the vessel may facilitate the use of detection instruments (e.g., optical instruments) positioned adjacent (e.g., above, below) the vessel, so that, for example, the surface portions and/or fluid portions within the vessel receive an appropriate distribution of light used for a variety of purposes, including, for example, metrics, photochemistry, and process control.

In some embodiments, the vessel is configured to withstand a certain internal pressure for conducting a reaction in the vessel. For instance, the vessel may be configured to withstand a pressure of least 1 psi, at least 1.5 psi, at least 2 psi, at least 2.5 psi, at least 3 psi, or at least 3.5 psi. The pressure in the vessel may be less than or equal to 4 psi, less than or equal to 3 psi, or less than or equal to 2 psi. Combinations of the above-referenced ranges are also possible (e.g., at least 1 psi and less than or equal to 4 psi). Combinations of the above-referenced ranges are also possible. A method of using a cartridge described herein may involve performing a reaction at one or more of the pressures described above in one or more vessels.

### Temperature Control Device

In some embodiments, at least one of the cassettes and/or at least one set of vessels is constructed and arranged to be heated (or cooled). In embodiments comprising two or more cassettes, a first and second cassette (or first and second set of vessels) may be constructed and arranged to be heated (or cooled) independently. For example, in some embodiments, a temperature control device is configured to apply a first temperature to a first cassette and a second temperature to a second module (e.g., simultaneously or sequentially). In some embodiments, individual vessels that are present in a cassette may be arranged to be heated or cooled independently. In some embodiments, the cartridge may comprise or interface with a temperature-control device. In certain embodiments, the cartridge may be in communication with the temperature-control device. In some embodiments, the cartridge can interface with a lid (e.g., a heated lid) that can be temperature-controlled. For example, in some embodiments, the cassette can interface with a lid including a temperature control device. In some embodiments, the lid covers at least one vessel. In certain embodiments, the lid (e.g., the temperature-controlled lid) forms a top portion of a vessel. Lids that can be temperature-controlled may be, in some cases, translucent or transparent. Advantageously, the temperature-controllable lid may be configured to allow optical measurements to be taken therethrough.

In some embodiments, cassette lids may be constructed from laser cut acrylic or injection molded acrylic (e.g., Acrylite H15). However, in some embodiments, the lid may be constructed from acrylics, polycarbonate, polypropylene, olefin polymers, polyethelyene, or polystyrene. In some embodiments, the heated lid is machined from aluminum and has a flexible resistive heater and thermal feedback inside. In some embodiments, the temperature control device comprises one or more thermal pads, thermoelectric components, and/or thermistors. Those skilled in the art would be capable of selecting suitable temperature control devices based upon the teachings of this specification.

In some embodiments, temperature control is accomplished using thermoelectric cooler (also known as TECs, Peltier heat pump, or solid state cooler). In some embodiments, temperature feedback is collected by using thermistors, resistive temperature detectors (RTDs), and thermocouples (type K). In some embodiments, the heating element in the heated lid is a flexible resistive heater and is only capable of heating and not cooling.

### Stored Reagents

As described herein, in some embodiments, at least one of the cassettes and/or vessels (or set of vessels) contains a reagent, such as a stored reagent. In certain embodiments, the stored reagent may be used for conducting a reaction, and in some cases may be a reactant. In some embodiments, the stored reagent may be for conducting a PCR reaction. In some embodiments, one or more of the following procedures (or reactions steps thereof) may be performed in a vessel: PCR, qPCR, RT-qPCR, RT/cDNA synthesis, Ligation, End-repair/End polishing (phosphorylation, A-tailing, End cleavage), Restriction enzyme digestion, nuclease cleavage, primer annealing, BER (base excision repair), and DNA denaturation.

In some embodiments, the stored reagent is a stored liquid reagent. In some embodiments, the stored liquid reagent includes a primer, a buffer, a wash reagent, and/or an alcohol (e.g., isopropanol, ethanol, methanol). In some embodiments, the primer is a PCR primer, a random hexamer, a RT specific primer, or a modified primer, such as a biotin labelled primer, phosphorylated primer, phosphorothioate bonded primer, a locked nucleic acid primer (LNA), or a fluorophore labelled primer. In some embodiments, the buffer is a Tris buffer, HEPES buffer, MOPS buffer, phosphate buffer, TE buffer, TBE buffer, lysis buffer, extraction buffer, PCR buffer, PBS buffer or wash buffer. In some embodiments, the wash reagent is water, ethanol, isopropanol, tris or a detergent solution. In some embodiments stored reagents may further comprise: PEG, Tris, betaine, glycerol free enzymes, dNTPs, salts, buffers, modified oligonucleotides, etc.

In certain embodiments, the stored reagent is a stored dried reagent. In some embodiments, the dried reagents are oligonucleotides, primers, synthetic template controls, fluorescent labelled probes, fluorescent dyes, buffers, master-mixes or enzymes.

In some embodiments, at least one of the cassettes and/or vessel (or set of vessels) contains one or more stored lyospheres. That is, in certain embodiments, the stored reagent is a stored lyosphere. For example, in one embodiment, at least one cassette and/or at least one vessel contains a single lyosphere. In another embodiment, at least one cassette and/or at least one vessel contains two or more lyospheres (e.g., two or more, three or more, or four or more lyospheres). In yet another embodiment, at least one cassette and/or at least one vessel contains a set of lyospheres. In some embodiments, at least a portion of the set of vessels contains at least one lyosphere disposed therein. For example, as shown illustratively in FIG. 8, cassette 1320 comprises a set of vessels 1340, each vessel containing a lyosphere (e.g., a vessel 1342 containing a lyosphere 1352, a vessel 1344 containing a lyosphere 1354, and/or a vessel 1346 containing a lyosphere 1356).

While a single lyosphere in each vessel is shown in FIG. 8, those skilled in the art would understand based on the present specification that one, or two or more, lyospheres may be present in each vessel in other embodiments. In certain embodiments, two or more cassettes comprise a set of lyospheres (e.g., one or more of cassettes 220, 222, 224, and/or 226 in FIGS. 4A and 4B may contain a set of lyospheres). In some embodiments, a cartridge comprises a first cassette comprising a first set of vessels containing stored lyospheres and a second module comprising a second set of vessels containing stored lyospheres. In some such embodiments, the first and second cassettes are not be in fluid communication with one another (e.g., prior to, or after, insertion of the cassettes in the cartridge/frame, and/or during storage). As described above, in some embodiments, the vessel(s) containing a stored reagent (e.g., liquid reagent) is/are sealed so as to reduce or prevent evaporation of the stored reagent, and/or to reduce or prevent contamination of the stored reagent.

In an exemplary embodiment, a cartridge comprises a first cassette comprising a first set of vessels, a second cassette comprising a second set of vessels, a first set of stored reagents for conducting a first reaction (e.g., a first PCR reaction) contained in the first set of vessels, and a second set of stored reagents for conducting a second reaction (e.g., a second PCR reaction) contained in the second set of vessels. In some such embodiments, as described above, the cartridge may be constructed and arranged to allow first and second reactions to be performed in parallel. In certain embodiments, the cartridge may be constructed and arranged to allow fluid communication between the channel system and at least one of the first and second cassettes during the first and/or second reactions, respectively. As described in more detail below, the channel system may include first and second sets of channels. The first set of channels may be in fluid communication with the first cassette comprising the first set of vessels, and the second set of channels may be in fluid communication with the second cassette comprising the second set of vessels. The first and second set of channels may be in fluid communication with one another via one or more valves. In some embodiments, lyospheres are obtained from commercial sources (e.g., from Biolyph LLC). In some embodiments, lyosphere sizes are in diameter range of 0.3 cm to 1 cm.

Any suitable material or combination of materials can be used to form the components of the system (e.g., modules, frame, cassette). In some embodiments, a rigid thermopolymer is used. The thermopolymer may be processed by any suitable method, such as injection molding. Non-limiting examples of materials include polymers (e.g., polypropylene, polyethylene, polystyrene, poly(acrylonitrile, butadiene, styrene), poly(styrene-co-acrylate), poly(methyl methacrylate), polycarbonate, polyester, poly(dimethylsiloxane), PVC, PTFE, PET, or blends of two or more such polymers), adhesives, metals including nickel, copper, stainless steel, bulk metallic glass, or other metals or alloys, or ceramics including glass, quartz, silica, alumina, zirconia, tungsten carbide, silicon carbide, or non-metallic materials such as graphite, silicon, or others. Other materials are also possible.

The components of the system (e.g., modules, frame, cassette) may have any suitable configuration. In some embodiments, the frame has a cross-sectional dimension (e.g., width, height) of at least 5 inches, at least 6 inches, at least 7 inches, at least 8 inches, at least 9 inches, at least 10 inches, at least 12 inches, or at least 20 inches; and/or less than or equal to 20 inches, less than or equal to 15 inches, less than or equal to 10 inches, or less than or equal to 5 inches. Combinations of the above-referenced ranges are also possible.

As described herein, in some embodiments, a cartridge comprises a channel system. Referring again to FIGS. 6A-6C, cartridge 1100 comprises channel system 1130 adjacent and non-integral to frame 1110. In certain embodiments, the channel system comprises at least one microfluidic channel (e.g., fluidic channel 1135 in FIGS. 6A-6B, fluidic channels 1137 and 1139 in FIG. 6C). In some cases, one or more microfluidic channels may be a common microfluidic channel. The term “common microfluidic channel” as used herein generally refers to a microfluidic channel associated with (e.g., in fluidic communication with, attached to) one or more secondary channels (e.g., one or more vessel channels), in which fluid can be transported to and from the common channel to the secondary channel(s). In some embodiments, the common microfluidic channel is connected to the secondary channel via a valve. For example, a valve may permit fluidic communication between a common microfluidic channel and a first channel (e.g., a first vessel channel connected to a vessel) and, upon switching of the valve, may permit fluidic communication between the common microfluidic channel and a second channel (e.g., a second vessel channel connected to a vessel). In certain embodiments, one or more fluidic components (e.g., a valve, a junction) may be fluidically connected to the common microfluidic channel, and/or to more than one common microfluidic channel(s) as described in more detail below. While much of the description herein relates to microfluidic channels, those skilled in the art would understand based upon the teachings of this specification that other fluidic conduits (e.g., channels, conduits, capillaries) may also be used.

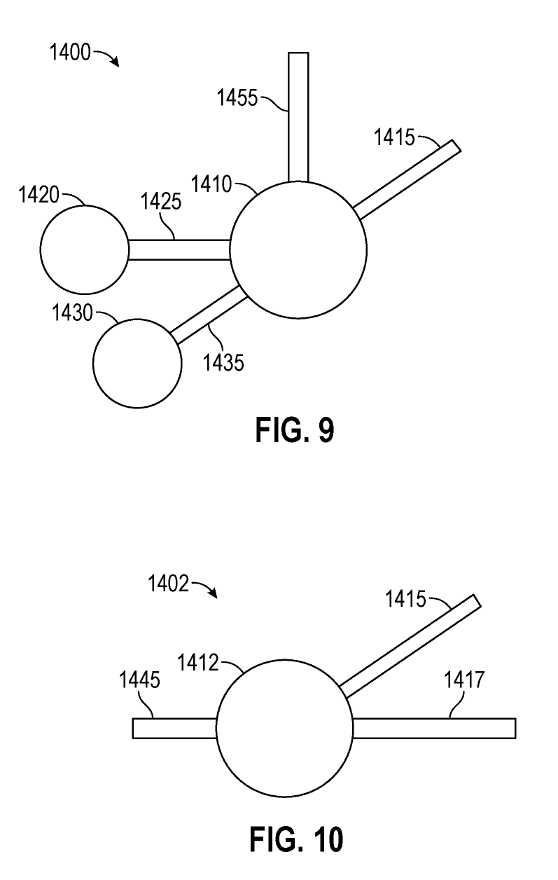

In some embodiments, a channel system comprises a common microfluidic channel, one or more valves, and one or more vessels or sets of vessels. In some cases, each vessels may be connected to a vessel channel. For example, as shown illustratively in FIG. 9, in some embodiments, a channel system 1400 comprises a common microfluidic channel 1415, a first vessel 1420 and a second vessel 1430. First vessel 1420 may be connected (e.g., fluidically connected) to a first vessel channel 1425 and second vessel 1430 may be connected to a second vessel channel 1435. In some embodiments, the channel system may comprise 1, 2, 4, 6, 8, or 10 or more (and/or less than or equal to 20, 15, 10, 5, or 4) common microfluidic channels, 1, 2, 4, 6, 8, or 10 or more (and/or less than or equal to 20, 15, 10, 5, or 4) sets of vessels, and/or vessel channels connected to each vessel. Each set of vessels may be positioned in a different cassette as described herein. In certain embodiments, each set of vessels may comprise 1, 2, 4, 6, 8, or 10 or more (and/or less than or equal to 20, 15, 10, 5, or 4) vessels. In some such embodiments, a first set of vessel channels and/or a second set of vessel channels includes at least 2, 4, 6, 8, or 10 vessel channels and/or less than or equal to 20, 15, 10, or 5 vessel channels. Combinations of the above-referenced ranges are also possible.

In some embodiments, each common microfluidic channel may be connected to a vessel or set of vessels via a valve. That is, in some embodiments, each common microfluidic channel and/or each vessel channel may extend from a valve. For example, referring again to FIG. 9, in some embodiments, common microfluidic channel 1415, first vessel channel 1425, and second vessel channel 1435 extend from valve 1410 (e.g., a rotary valve). In some embodiments, 1, 2, 4, 6, 8, or 10 or more vessel channels and one or more secondary channels such as channel 1455 (e.g., one or more sample channels, one or more waste channels, one or more inlet channels, etc.) may extend from the valve. In some embodiments, the valve may be operated (e.g., switched, rotated) such that the common microfluidic channel and the first vessel channel, the common microfluidic channel and the second vessel channel, or the first vessel channel and the second vessel channel, are in fluidic communication with one another.

In some embodiments, two or more common microfluidic channels extend from the valve. For example, as shown illustratively in FIG. 10, in some embodiments, a channel system 1402 comprises a valve 1412, common microfluidic channels 1415 and 1417 connected to valve 1412, and an inlet channel 1445. Secondary channels (e.g., vessel channels) and/or vessels may be connected (directly or indirectly) to each of the common microfluidic channels (not shown). In some such embodiments, valve 1412 may permit fluidic communication between inlet channel 1445 and common microfluidic channel 1415 (but not common microfluidic channel 1417) and, upon switching the valve 1412, the valve may permit fluidic communication between inlet channel 1445 and common microfluidic channel 1417 (but not common microfluidic channel 1415). In some embodiments, common microfluidic channel 1415 is fluidically connected to a first cassette (e.g., including a first set of vessels) and common microfluidic channel 1417 is connected to a second cassette (e.g., including a second set of vessels). As such, the inlet channel may be fluidically connected at a downstream end to one or more cassettes (or one or more vessels of one or more cassettes) via the common microfluidic channel. In another example, a user may insert a cassette into a cartridge as described herein and, upon insertion, the cassette is in fluidic communication with the inlet channel at an upstream and such that a fluid present in the cassette may be directed, via the valve, to one or more common microfluidic channels.

In one set of embodiments, as shown illustratively in FIG. 11, a channel system 1404 includes a first set of channels 1406 and a second set of channels 1408. The first set of channels may be used for conducting a first set of reactions (e.g., a first PCR reaction) and the second set of channels may be used for conducting a second set of reactions (e.g., a second PCR reaction). The first set of channels 1406 may include a first set of vessel channels 1422 connected to a first set of vessels 1440; and the second set of channels 1408 may include a second set of vessel channels 1427 connected to a second set of vessels 1442.

In some embodiments, first set of vessels 1440 comprises a plurality of vessels (and vessel channels connected to the vessels), each vessel channel extending from valve 1410. Valve 1410 may be connected to common microfluidic channel 1415, which may be used for introducing reagents/fluids into, and/or removing reagents/fluids from, channel system 1406. In certain embodiments, second set of vessels 1442 comprises a plurality of vessels (and vessel channels connected to the vessels), each vessel extending from valve 1414. Valve 1414 may be connected to common microfluidic channel 1417, which may be used for introducing reagents fluids into, and/or removing reagents/fluids from, channel system 1408.

As described herein, the vessels may be part of a cassette that is inserted or otherwise a part of a cartridge. For example, in one set of embodiments, first set of vessels 1440 (e.g., vessels 1420 and 1430) shown in FIG. 11 may be a part of cassette 1122 as shown in FIG. 6C, and second set of vessels 1442 may be a part of cassette 1124 as shown in FIG. 6C. Channel system 1130 (FIG. 6C) may include channel system 1404 of FIG. 11. For example, channel 1139 (FIG. 6C) may be one of vessel channels 1422 (FIG. 11), and channel 1137 (FIG. 6C) may be one of vessel channels 1427 (FIG. 11). Other configurations are also possible.

Various configurations of channels and valves may be possible in a channel system described herein. For instance, in some embodiments a channel system comprises valve 1412 and common microfluidic channels 1415 and 1417 extending from valve 1412. As shown illustratively in FIG. 11, the first and second sets of vessel channels may be separated from one another by at least one valve and/or by at least one common microfluidic channel. That is, in some embodiments, one or more common microfluidic channels may be positioned between a first set of vessel channels and a second set of vessel channels. In certain embodiments, a common microfluidic channel may be positioned between a first valve and a second valve. For example, common microfluidic channel 1415 is shown illustratively in FIG. 11 as being positioned between valve 1412 and valve 1410. In certain embodiments, common microfluidic channel 1417 is positioned between valve 1412 and valve 1414.

In some embodiments, the channel system comprises one or more channels (or set of channels) for conducting one or more reactions (e.g., a first PCR reaction, a second PCR reaction, etc.). For example, referring again to FIG. 11, in some embodiments, the first set of channels 1406 including first set of vessel channels 1422 connected to the first set of vessels 1440 are configured for conducting a first reaction, and the second set of channels 1408 including second set of vessel channels 1427 connected to the second set of vessels 1442 are configured for conducting a second reaction. The first and second reactions may be conducted in parallel, or sequentially.

In some embodiments, the channel system comprises secondary channels such as a waste channel connected to a waste vessel, a sample inlet channel connected to a sample well, and/or an output channel connected to an output well. Referring again to FIG. 11, in some embodiments, valve 1410 may be connected to sample inlet channel 1455, output channel 1460, and/or waste channel 1462. The sample inlet channel, may be connected to one or more sample wells (e.g., as part of a sample cassette 1490, in fluidic communication with sample inlet channel 1455). The output channel may be connected to one or more output wells (e.g., as part of a output cassette 1495, in fluidic communication with output channel 1460). The waste channel may be connected to one or more waste wells (e.g., as part of a waste cassette, not shown). In certain embodiments, valve 1414 may be connected to sample inlet channel 1457, output channel 1465, and/or waste channel 1467. The sample inlet channel may be connected to one or more sample wells (e.g., as part of the sample cassette, not shown). The output channel may be connected to one or more output wells (e.g., as part of the output cassette, not shown). The waste channel may be connected to one or more waste wells (e.g., as part of a waste cassette, not shown). In certain embodiments, valve 1412 is connected to one or more fluid inlet channels (e.g., fluid inlet channels 1445 and 1447) that may transport one or more fluids/reagents to the channel systems.

In some embodiments, one or more valves of the channel system is a rotary valve. In certain embodiments, one or more valves of the channel system comprises a raised feature configured to facilitate the flow of a fluid between the common microfluidic channel and another channel. In some cases, the one or more valves of the channel system comprises a seal.

In some embodiments, the valve is constructed and arranged to be in fluidic communication with the common microfluidic channel and one secondary channel. For example, the valve may be actuated such that the common microfluidic channel may be in fluidic communication with a desired channel upon actuation of the valve.

In general, the channel systems or portions thereof described herein may be used to control the direction and/or volume of a fluid. The methods described herein may be useful, for example, for mixing and/or reacting two or more reagents and/or fluids in controlled volumes. In some cases, at least one vessel may contain a reagent (e.g., a stored reagent) therein. Two or more vessels may contain different reagents depending on the desired reaction (e.g., a first reagent for conducting a first reaction and a second reagent for conducting a second reaction). The two or more vessels may be in fluidic communication with the channel system such that fluids can be transported between each of the vessels. In some embodiments, the order of the mixing and/or reactions may be controlled (e.g., by controlling and/or alternating the direction of fluid flow).

In some cases, at least a portion of the fluid transferred to the first vessel may be exposed to (e.g., reacted with) a first stored reagent present in the first vessel. In some embodiments, the resulting fluid (e.g., the reacted fluid) may be transported from a first vessel to a second vessel. In certain embodiments, upon entering the second vessel, the fluid is exposed to (e.g., reacted with) the second reagent. In some embodiments, a sample and/or reactant present in the fluid reacts with the first reagent and/or the second reagent.

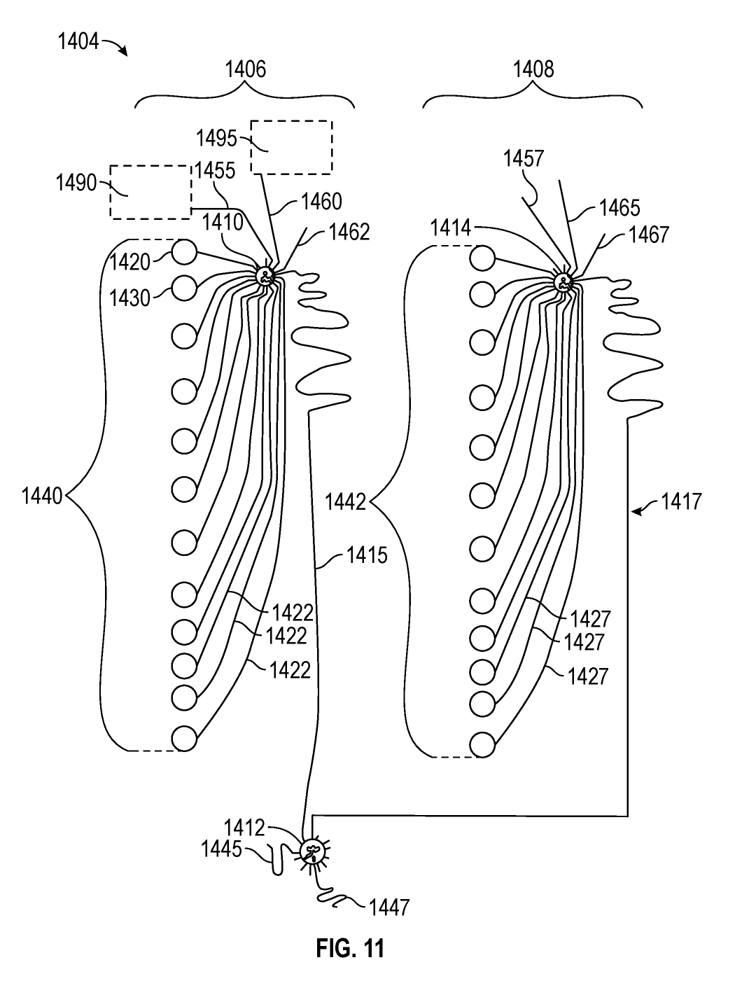

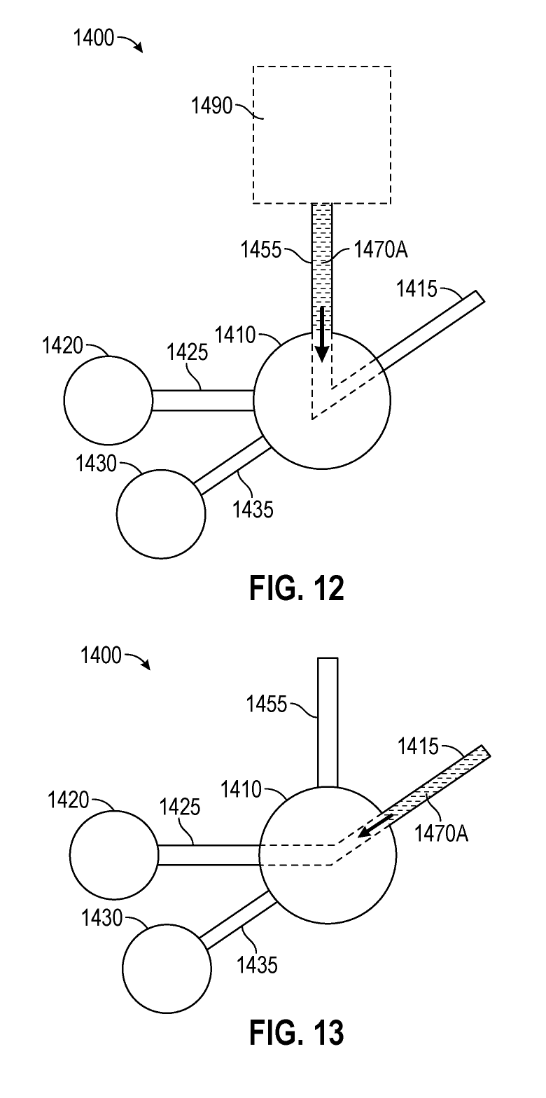

In some embodiments, the transfer of the fluid between vessels may be controlled (e.g., by actuating a valve disposed between the common microfluidic channel and one or more vessel channels). For example, in certain embodiments, the common microfluidic channel may be utilized to facilitate the transfer of (e.g., the direction of flow of) the fluid. In some such embodiments, at least a portion of the reacted fluid (e.g., after reacting with the first stored reagent) may be transported from the first vessel to the common microfluidic channel and subsequently transported to the second vessel, such that a portion of the fluid may react with the second stored reagent present in the second vessel. In some embodiments, a series of reactions (e.g., PCR reaction steps) may be conducted sequentially by flowing a fluid between the common microfluidic channel and two or more vessels. In some such embodiments, the valve may be actuated such that the common microfluidic channel is in fluidic communication with the sample inlet channel, the first vessel channel, or the second vessel channel to facilitate the transfer/flow of at least a portion of the fluid between the sample inlet channel, the first vessel, and/or the second vessel. This process is illustrated in FIGS. 12-21, which generically depict a channel system 1400 in different fluid flow configurations

For example, in some embodiments, as illustrated in FIG. 12, a fluid 1470A (e.g., a sample) may be introduced into the channel system via sample inlet channel 1455 (e.g., connected to and in fluidic communication with sample cassette 1490). The fluid may be flowed in a direction indicated by the arrow in FIG. 12. In some cases, fluid 1470A may be provided or introduced by a user into a sample well connected to sample inlet channel 1455. The fluid may be transferred to common microfluidic channel 1415 by flowing the fluid from sample inlet channel 1455 through valve 1410 (positioned between the common microfluidic channel and a set of vessels, and actuated such that the common microfluidic channel and the sample inlet channel are in fluidic communication with each other).

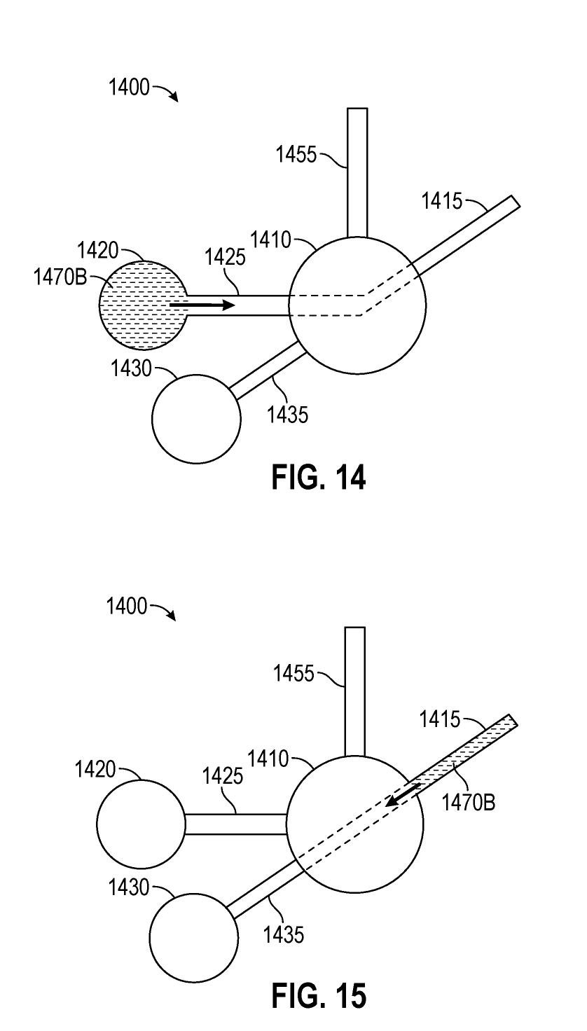

In some embodiments, as illustrated in FIG. 13, at least a portion of the fluid present in common microfluidic channel 1455 may be transported (e.g., flowed), through valve 1410 to first vessel 1420 via first vessel channel 1425. First vessel 1420 may be constructed and arranged for housing or conducting a first reaction (e.g., a first part of a PCR reaction, a chemical, and/or biological reaction). In some such embodiments, valve 1410 may be actuated such that the common microfluidic channel 1455 and first vessel channel 1425 are in fluidic communication with one another to allow transport of fluid 1470A to first vessel 1420 (FIG. 13). In some embodiments, a first reaction may take place in first vessel 1420 (e.g., between a sample component and a first reagent positioned within the first vessel). After the first reaction takes place in the first vessel, the reaction product may be transported back to the first vessel channel and subsequently to common microfluidic channel via the valve. For example, in some embodiments, as illustrated in FIG. 14, fluid 1470B (e.g., the fluid now comprising the reaction product) may be transported to common microfluidic channel 1415 from vessel 1420. In some embodiments, the fluid may be flowed in a particular direction. For example, in some cases, the fluid may be flowed in a first direction (e.g., a first direction in the microfluidic channel (e.g., in a common microfluidic channel) towards the valve as indicated by the arrow in FIG. 13). In certain embodiments, the fluid may be flowed in a second direction opposite the first direction (e.g., a second direction in the microfluidic channel (e.g., in a common microfluidic channel) away from the valve). In some embodiments, flowing the fluid in opposite directions (e.g., before and after actuating the valve) facilitates the transfer of the fluid between the common microfluidic channel and two or more vessel channels.

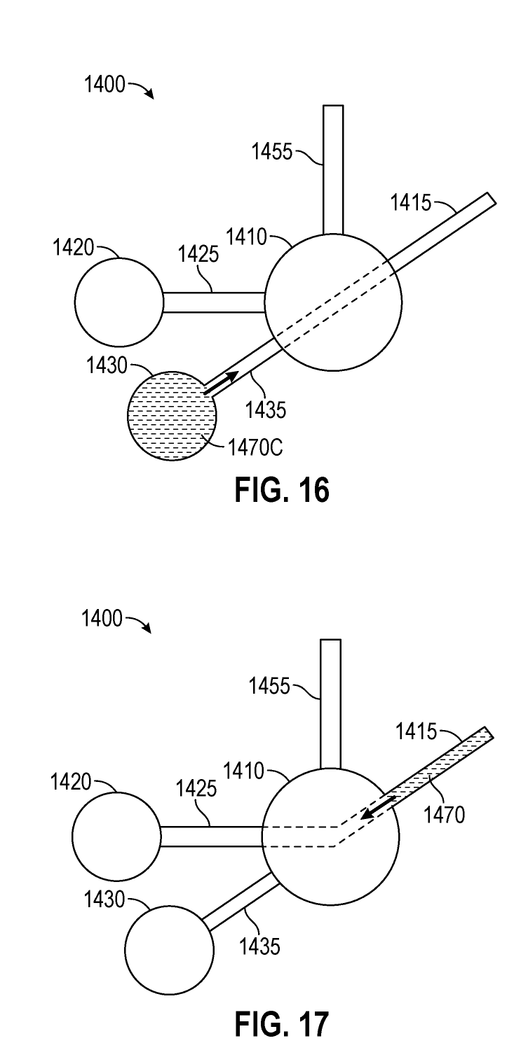

In certain embodiments, the reaction product (e.g., fluid 1470B comprising the reaction product in FIG. 15) may be transported to the second vessel via the second vessel channel. The second vessel may, for example, be constructed and arranged for conducting a second reaction (e.g., a second part of a PCR reaction), independent of the first reaction. As illustrated in FIG. 15, in some such embodiments, valve 1415 may be actuated such that common microfluidic channel 1415 and second vessel channel 1435 are in fluidic communication with one another, and fluid 1470B may be transported from common microfluidic channel 1415 to second vessel 1430 via valve 1415 and into second vessel channel 1435 (FIG. 16) In certain embodiments, a second reaction may take place in second vessel 1430 (e.g., between a sample component and a second reagent positioned within the second vessel). After the second reaction, the reaction product (e.g., present in fluid 1470C) may be transported from second vessel 1430 to second vessel channel 1435 and back to common microfluidic channel 1415 via valve 1410. The same process can be used to transport fluids into various vessels for conducting various reactions (e.g., sequential reactions). Advantageously, numerous reaction and mixing steps may be facilitated through the use of a common microfluidic channel and a set of vessels as described herein.

A fluid may remain in one or more vessels and/or the common microfluidic channel for any suitable amount of time (e.g., up to 30 minutes, up to 1 hour, up to 2 hours, up to 3 hours, up to 4 hours, up to 5 hours, up to 6 hours or more).

In some embodiments, at least a portion of the fluid (e.g., the reaction product of a reaction, or the reaction product of two or more sequential reactions) may be transported to an output channel (e.g., for collection and/or analysis) and/or to a waste channel. For example, in some embodiments, at least a portion of the fluid may be transferred to the common microfluidic channel and subsequently transferred to the output channel (e.g., the output channel connected to one or more output wells). In some embodiments, at least a portion of the fluid present in the common microfluidic channel is transferred into the output channel or a waste channel (e.g., a waste channel connected to a waste cassette). In certain embodiments, substantially all of the fluid remaining in the common microfluidic channel is transferred into the output channel and/or the waste channel. In certain embodiments, at least a portion of the fluid present in the common microfluidic channel which is not transferred into the first and/or second vessel may be flowed into a waste channel.

In certain embodiments, the amount (e.g., volume) of fluid mixed and/or reacted may be controlled. In some cases, it may be desirable to add a particular volume of a fluid to a vessel (e.g., to facilitate a controlled reaction with a reactant and/or sample). In some embodiments, the particular volume of fluid may be isolated within a vessel and/or the common microfluidic channel. Advantageously, the systems and methods described herein may facilitate the transfer of known/predetermined volumes of fluids to vessels and/or channels. For example, at least 1 microliter, at least 2 microliters, at least 5 microliters, or at least 10 microliters may be transferred between the first vessel and the common microfluidic channel. Advantageously, desired volumes of reactants and/or samples may be reacted with one or more fluids via the methods described herein, such that a particular reaction step may be accurately conducted.

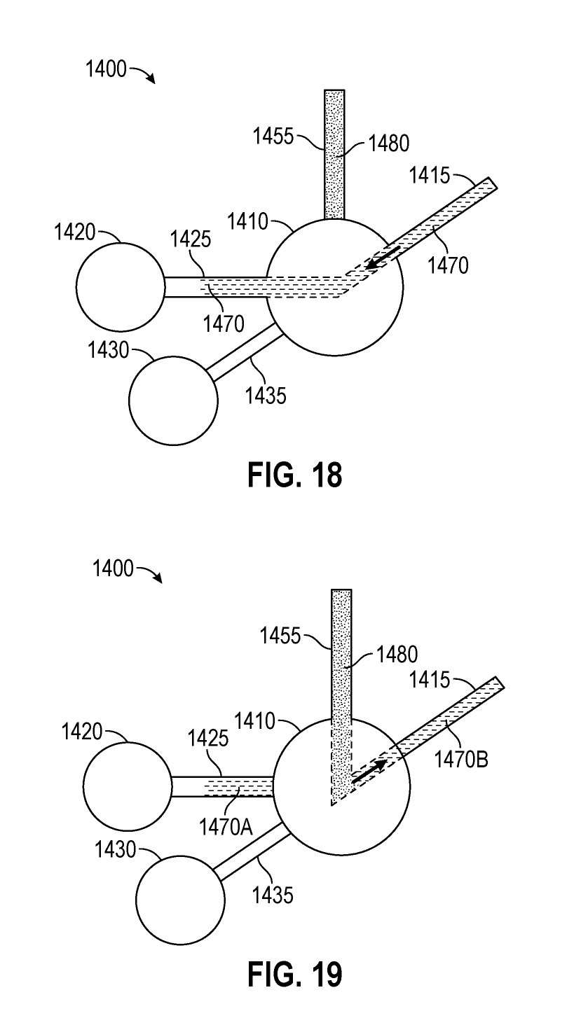

In some embodiments, a second fluid (e.g., a fluid immiscible with the first fluid) may be utilized to direct (e.g., push) a particular volume of the first fluid between the common microfluidic channel and one or more vessel channels. In an exemplary embodiment, as shown in FIG. 17, (first) fluid 1470 is disposed within common microfluidic channel 1415. At least a portion of fluid 1470 may be transported to first vessel 1420 by actuating the valve 1410 such that first vessel channel 1425 and common microfluidic channel 1415 are in fluidic communication. As shown in FIG. 18, at least a portion of fluid 1470 may be transported into at least a portion of first vessel channel 1425. In some such embodiments, the volume of the portion of fluid 1470 transported into at least a portion of first vessel channel 1425 may be selected. For example, a pump (e.g., a syringe pump) in fluidic communication with common microfluidic channel 1415 may be actuated such that a known volume of fluid 1470 is disposed within first vessel channel 1425. Referring again to FIG. 11, in certain embodiments, the pump may be fluidically connected to inlet channel 1447 and valve 1412 may be actuated such that inlet channel 1447 and common microfluidic channel 1415 are in fluidic communication.

Referring again to FIG. 18, a second fluid 1480 may be provided to sample inlet channel 1455. As shown in FIG. 19, second fluid 1480 may be transported to common microfluidic channel (e.g., effectively displacing fluid 1470 further into the common microfluidic channel) by actuating valve 1410 such that sample inlet channel 1455 and common microfluidic channel 1415 are in fluidic communication. Second fluid 1480 may be flowed in a first direction (as indicated by the arrow in FIG. 19). In some embodiments, this second fluid can partition the first fluid 1470 into at least portions, e.g., portion 1470A and 1470B as shown illustratively in FIG. 19. The fluid portions may be in the form of fluid plugs (e.g., a first fluid plug and a second fluid plug) that are separated by at least the second fluid, which in some instances may be immiscible with the first fluid.

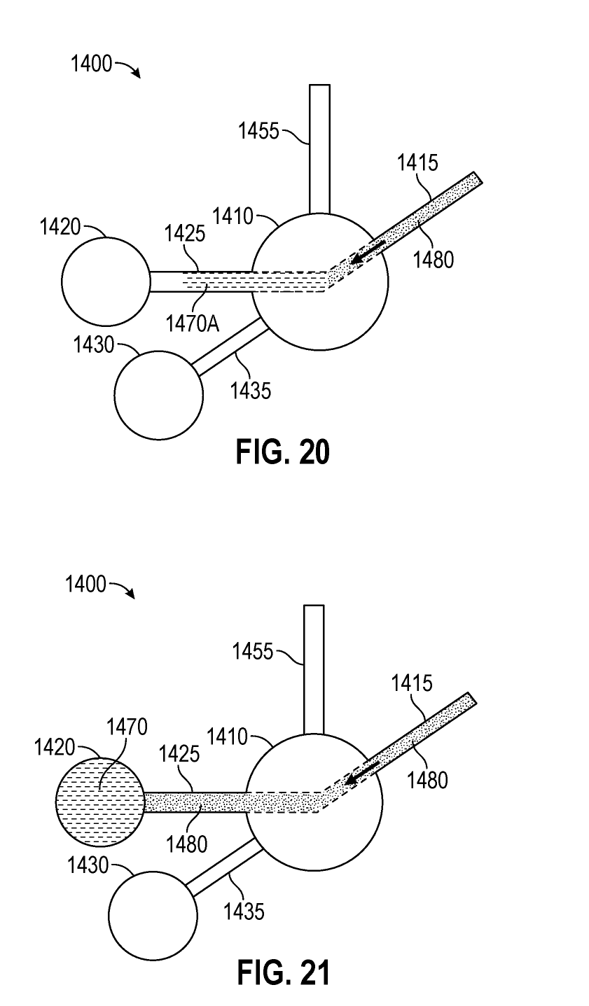

Referring now to FIG. 20, valve 1410 may be actuated such that common microfluidic channel 1415 and first vessel channel 1425 are in fluidic communication. At least a portion of second fluid 1480 may be transported (flowed in a second direction opposite the first direction as indicated by the arrow in FIG. 20) towards first vessel channel 1425 such that fluid 1470A is displaced. In some such embodiments, a desired volume of fluid 1470A is transported to first vessel 1420 (e.g., as depicted in FIG. 21) by second fluid 1480. In some embodiments, at least a portion of the second fluid is also introduced into the first vessel. However, in other embodiments, essentially none of the second fluid is introduced into the vessel.

While much of the description above relates to the transfer of a desired volume of fluid between the common microfluidic channel and the first vessel channel, those skilled in the art would understand based upon the teachings of the specification that a series of steps such as those described above may be utilized to facilitate the transfer of desired volumes of fluids between the common microfluidic channel and one or more vessels, one or more vessel channels, one or more output channels, and/or one or more waste channels. For example, in some embodiments, it may be desirable to have a first volume of a fluid transported to the first vessel, and a second volume of fluid, different than the first volume, transported to the second vessel. In some such embodiments, the steps described above may be utilized to facilitate such a transfer of fluid volume(s).

In some cases, the second fluid described above is immiscible (e.g., the second fluid is air) with the first fluid (e.g., an aqueous fluid). In some embodiments, the second fluid may be used to push the first fluid in a particular direction (e.g., a direction in the common microfluidic channel) and/or into a channel or other component (e.g., the common microfluidic channel, a vessel channel, an output channel, a waste channel). In certain embodiments, the second fluid may be used to keep fluids separate (e.g., as fluid plug(s)). For example, in some cases, the second fluid may be present in a channel and disposed between two fluid portions (e.g., a first fluid and a third fluid). In some such embodiments, the first fluid and the third fluid maybe the same or different.

As described above, the second fluid, in some embodiments, facilitates the flow of the controlled volume of the first fluid. In some embodiments, the controlled volume has a volume of at least about 5 μL (e.g., at least about 10 μL, at least about 20 μL, at least about L, at least about 40 μL, at least about 50 μL, at least about 80 μL, at least about 100 μL, at least about 200 μL) and/or less than or equal to 500 μL (e.g., less than or equal to 400 μL, less than or equal to 300 μL, less than or equal to 200 μL, less than or equal to 100 μL, less than or equal to 80 μL, less than or equal to 60 μL, less than or equal to 40 μL, less than or equal to 20 μL, less than or equal to 10 μL, less than or equal to 5 μL, less than or equal to 2 μL). Combinations of the above-referenced ranges are also possible. Other volumes are also possible.

In general, the internal volume of one or more vessel channels and the common microfluidic channel may be known and may be used to partition fluids of particular volumes. For example, in some such embodiments, the dimensions (e.g., length, cross-sectional dimensions) of a particular channel in which a fluid is disposed may be utilized to determine the volume of the fluid disposed within the channel. As an illustrative example, in a particular embodiment, the first vessel channel may have an internal volume of 2 microliters. In such an embodiment, if 1 microliter of fluid is desired to be added to the first vessel, pressure may be applied to the common microfluidic channel (in fluidic communication with the first vessel channel) such that the fluid is disposed halfway into the first vessel channel. The valve may be actuated as described above and a second fluid (e.g., a fluid immiscible with the first fluid such as air) may be introduced into the common microfluidic channel and redirected into the first vessel channel such that 1 μL of the first fluid is displaced by the second fluid and transported into the first vessel. In some such embodiments, at least a portion of the second fluid may be transported into the first vessel. However, in certain embodiments, substantially none of the second fluid is transported into the first vessel (e.g., a pressure is applied to the common microfluidic channel such that the first fluid is transported to the first vessel but substantially none of the second fluid is transported to the first vessel).

Fluids can be transferred (e.g., transport, flowed, displaced) into a microfluidic channel (e.g., common microfluidic channel, vessel channel, etc.), vessel, or cassette using any suitable component, for example, a pump, syringe, pressurized vessel, or any other source of pressure. Alternatively, fluids can be pulled into the microfluidic channel, vessel, or cassette by application of vacuum or reduced pressure on a downstream side of the channel or device. Vacuum may be provided by any source capable of providing a lower pressure condition than exists upstream of the channel or device. Such sources may include vacuum pumps, venturis, syringes and evacuated containers. It should be understood, however, that in certain embodiments, methods described herein can be performed with a changing pressure drop across an inlet and an outlet of the microfluidic device by using capillary flow, the use of valves, or other external controls that vary pressure and/or flow rate.

In some embodiments, flowing the fluid (e.g., the first fluid, the second fluid) or at least a portion of the fluid comprises applying a pressure to the common microfluidic channel such that at least a portion of a first fluid enters the vessel channel. In certain embodiments, flowing the fluid comprises applying a pressure to the common microfluidic channel such that at least a portion of a first fluid is transferred from (or to) the vessel channel to (or from) the vessel. In some embodiments, the pressure is a positive pressure. In certain embodiments, the pressure is a negative or reduced pressure. In certain embodiments, a volume of the fluid present in the vessel channel and/or a vessel connected to the vessel path is controlled by the pressure applied to the common microfluidic channel.

In some embodiments, as described above, the valve may direct the transfer from the fluid between the various channels (e.g., between the first vessel channel and the common microfluidic channel, between the first vessel channel and the waste channel, between the sample inlet and the microfluidic channel, etc.). In some embodiments, flowing at least a portion of the fluid (e.g., the first fluid, the second fluid) comprises actuating a/the valve such that the common microfluidic channel is in fluidic communication with the vessel channel (e.g., the first vessel channel, the second vessel channel).

In some embodiments, the first fluid may be a liquid sample, a reagent, water, a buffer, or the like. In certain embodiments, the second fluid may be a liquid sample, a reagent, water, a buffer, or the like. In some cases, the second fluid may be immiscible with the first fluid. In some such embodiments, the second fluid may be a gas (e.g., air, nitrogen). In some embodiments, fluids may be selected from: mineral oil, silicone oil, ethanol, tris, water, PEG solution, fluorophore, fluorocarbon or fluorosilicon-based compound, e.g., perfluoro.

In certain embodiments, one or more channels of the channel system has a particular average cross-sectional dimension. The “cross-sectional dimension” (e.g., a diameter) of the channel is measured perpendicular to the direction of fluid flow. In some embodiments, the average or largest cross-sectional dimension of the channel is less than or equal to about 2 mm, less than or equal to about 1 mm, less than or equal to about 800 microns, less than or equal to about 600 microns, less than or equal to about 500 microns, less than or equal to about 400 microns, or less than or equal to about 300 microns. In certain embodiments, the average or largest cross-sectional dimension of the channel is greater than or equal to about 50 microns, greater than or equal to about 100 microns, greater than or equal to about 150 microns, greater than or equal to about 200 microns, greater than or equal to about 250 microns, greater than or equal to about 300 microns, greater than or equal to about 400 microns, greater than or equal to about 500 microns, greater than or equal to about 600 microns, greater than or equal to about 800 microns, or greater than or equal to about 1 mm. Combinations of the above-referenced ranges are also possible (e.g., between about 250 microns and about 2 mm, between about 400 microns and about 1 mm, between about 300 microns and about 600 microns). Other ranges are also possible. In some cases, more than one channel or capillary may be used. “Microfluidic channels” refer to channels having an average cross-sectional dimension of less than 1 mm.

One or more microfluidic channels of the channel system may have any suitable internal volume. In some embodiments, the internal volume of the channel (e.g., microfluidic channel, common channel, vessel channel) may be at least 0.1 microliters, at least 0.5 microliters, at least 1 microliter, at least 2 microliters, at least 5 microliters, at least 7 microliters, at least 10 microliters, at least 12 microliters, at least 15 microliters, at least 20 microliters, at least 30 microliters, or at least 50 microliters. In certain embodiments, the internal volume of the microfluidic channel may be less than or equal to 200 microliters, less than or equal to 150 microliters, less than or equal to 100 microliters, less than or equal to 80 microliters, less than or equal to 70 microliters, less than or equal to 50 microliters, less than or equal to 25 microliters, less than or equal to 10 microliters, or less than or equal to 5 microliters. Combinations of the above-referenced ranges are also possible (e.g., between 1 microliter and 10 microliters). Other ranges are also possible. In some embodiments, an internal volume of the first vessel channel is less than an internal volume of the second vessel channel.

One or more channels (e.g., microfluidic channels) of the channel system can have any suitable cross-sectional shape (circular, oval, triangular, irregular, trapezoidal, square or rectangular, or the like). A microfluidic channel may also have an aspect ratio (length to average cross sectional dimension) of at least 2:1, more typically at least 3:1, 5:1, or 10:1 or more. A fluid (e.g., a sample) within the channel may partially or completely fill the channel.

In some embodiments, the one or more channels (e.g., microfluidic channels) may have a particular configuration. In certain embodiments, at least a portion of one or more microfluidic channels may be substantially linear in the direction of fluid flow. In some embodiments, substantially all of one or more microfluidic channels is substantially linear in the direction of fluid flow. In some embodiments, at least a portion of one or more microfluidic channels (e.g., a common microfluidic channel) may be curved, bent, serpentine, staggered, zig-zag, spiral, or combinations thereof. Advantageously, the use of a non-linear microfluidic channel (e.g., a serpentine common channel) permits an increased holding volume of the channel per unit length of the cartridge measured in the average direction of fluid flow as compared to a linear microfluidic channel.

A channel system or portions thereof (e.g., a valve, a microfluidic channel) described herein can be fabricated of any suitable material. Non-limiting examples of materials include polymers (e.g., polypropylene, polyethylene, polystyrene, poly(acrylonitrile, butadiene, styrene), poly(styrene-co-acrylate), poly(methyl methacrylate), polycarbonate, polyester, poly(dimethylsiloxane), PVC, PTFE, PET, or blends of two or more such polymers, adhesives, or metals including nickel, copper, stainless steel, bulk metallic glass, or other metals or alloys, or ceramics including glass, quartz, silica, alumina, zirconia, tungsten carbide, silicon carbide, or non-metallic materials such as graphite, silicon, or others. In some embodiments, pressure-sensitive adhesives, such as acrylics and silicone adhesives, are used. In some instances, thermosetting adhesives can also be used. In certain embodiments, the channels can be created by injection molding the channel into a substrate and capping that substrate with a top resin layer that is fastened by pressure sensitive adhesive, thermosetting adhesive, laser welding, ultrasonic bonding, or any other bonding method that seals the capping layer to the substrate near the channel and forms a seal.

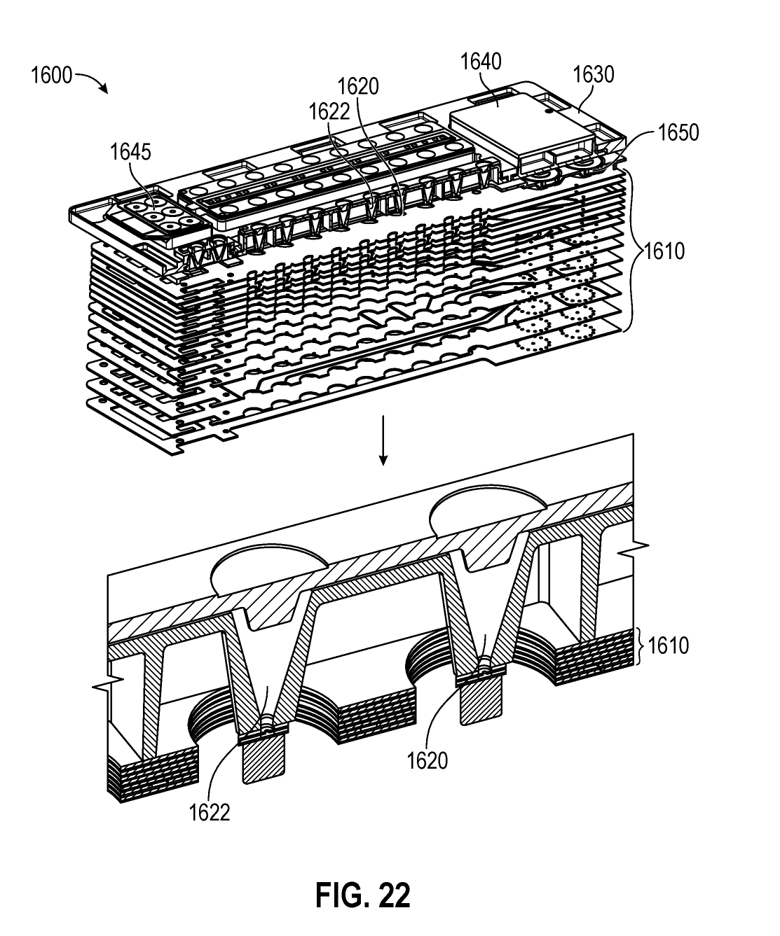

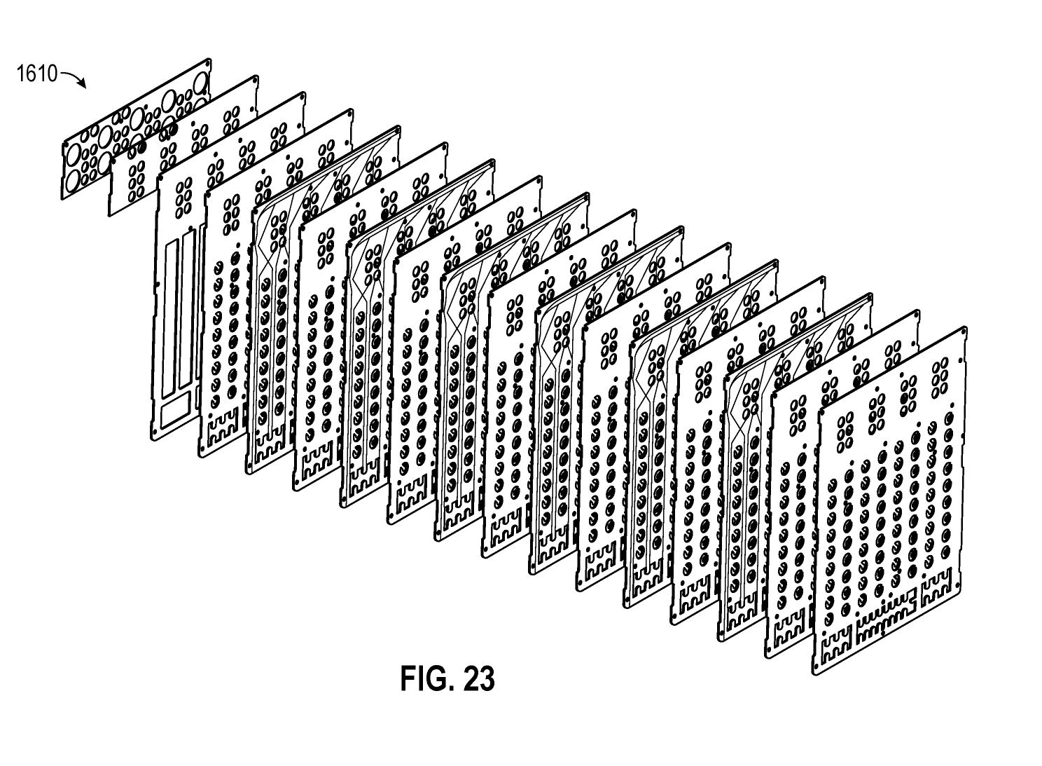

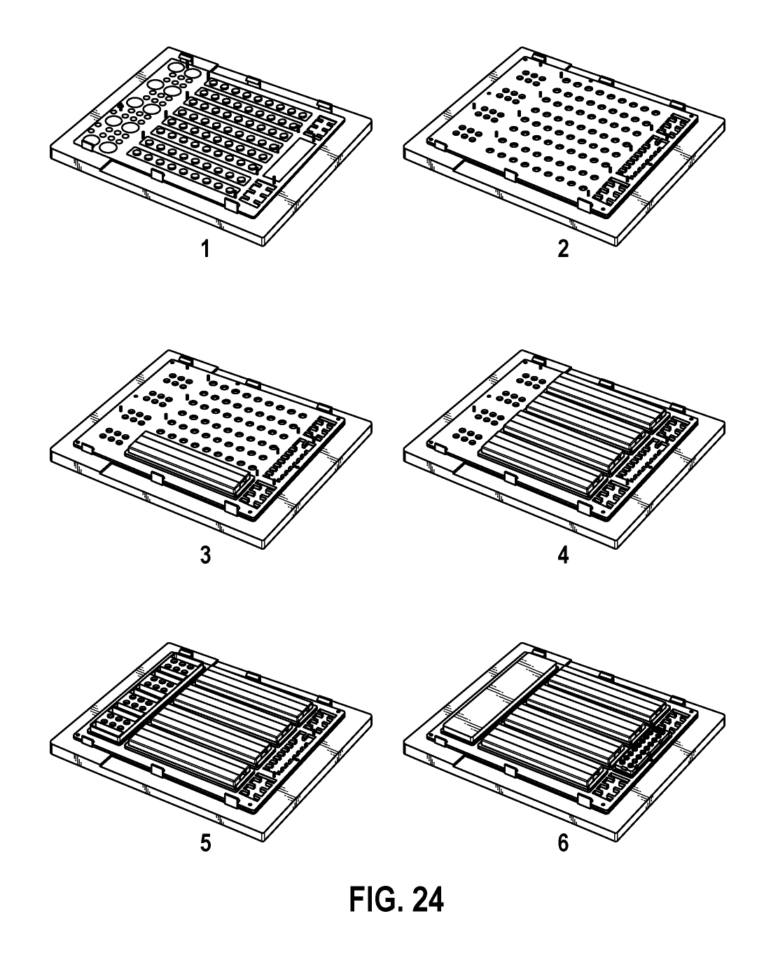

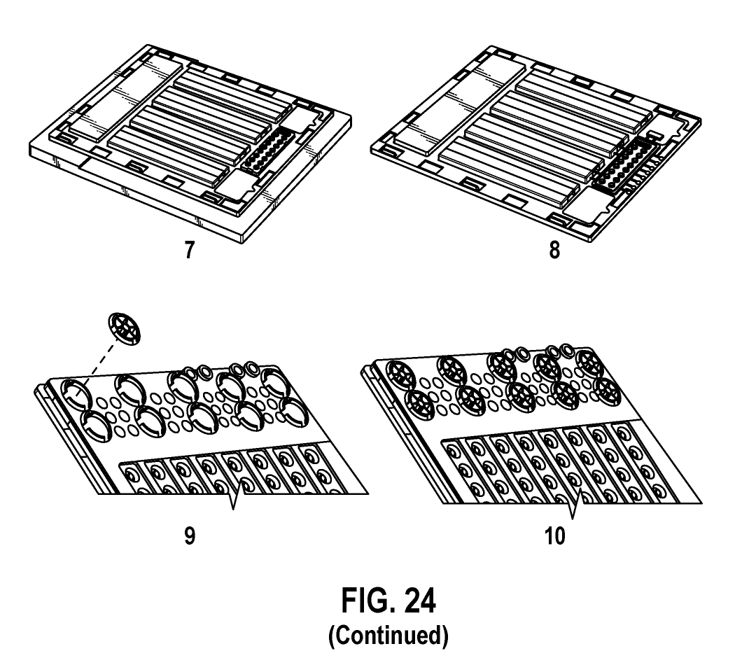

In some embodiments, the channel system or portions thereof is formed by a plurality of layers, such as alternating polymer and adhesive layers, forming the microfluidic channels therein. In an exemplary embodiment, as shown in FIGS. 22-23, cassette 1600 comprises a set of vessels (e.g., comprising vessel 1620 and vessel 1622) and channel system 1610, which is fabricated by assembling a plurality of alternating polymer layers and adhesive layers, each layer comprising a pattern that forms a plurality of channels when assembled. Cartridge 1600 may comprise frame 1630, with cassettes 1640 and 1645 inserted into openings within the frame. In some embodiments, cartridge 1600 comprises one or more valves (e.g., valve 1650) constructed and arranged to be in fluidic communication with a common microfluidic channel of channel system 1610. In one set of embodiments, the assembly comprises alternating layers of pressure sensitive adhesive and polyester resin layers. For example, in some instances with respect to FIG. 23, the darker layers are adhesive layers and the lighter layers are resin. The pressure sensitive adhesive may be cured by pressing the layers together in a press and waiting a few seconds for the bond to occur. If a thermosetting adhesive is used, then heat and pressure may be used to cure the adhesive. In some embodiments, the plurality of layers are as shown in layers 1-10 depicted in FIG. 24.

Other methods for forming microfluidic channels are known in the art and include, for example, microfabrication, molding, casting, chemical etching, photolithography, and combinations thereof.

### Amplification (AMP) Methods

Described herein are methods of determining the nucleotide sequence contiguous to a known target nucleotide sequence. The methods may be implemented in an automated fashion using the systems disclosed herein. Traditional sequencing methods generate sequence information randomly (e.g., “shotgun” sequencing) or between two known sequences which are used to design primers. In contrast, certain of the methods described herein, in some embodiments, allow for determining the nucleotide sequence (e.g., sequencing) upstream or downstream of a single region of known sequence with a high level of specificity and sensitivity.

In some embodiments, the systems provided herein may be configured to implement, e.g., in an automated fashion, a method of enriching specific nucleotide sequences prior to determining the nucleotide sequence using a next-generation sequencing technology. In some embodiments, methods provided herein can relate to enriching samples comprising deoxyribonucleic acid (DNA). In some embodiments, methods provided herein comprise: (a) ligating a target nucleic acid comprising the known target nucleotide sequence with a universal oligonucleotide tail-adapter; (b) amplifying a portion of the target nucleic acid and the amplification strand of the universal oligonucleotide tail-adapter with a first adapter primer and a first target-specific primer; (c) amplifying a portion of the amplicon resulting from step (b) with a second adapter primer and a second target-specific primer; and (d) transferring the DNA solution to a user. In some embodiments, one or more steps of the methods may be performed within different vessels of a cartridge provided herein. In some embodiments, microfluidic channels and valves in the cartridge facilitate the transfer of reaction material/fluid from one vessel to another in the cartridge to permit reactions to proceed in an automated fashion. In some embodiments, a DNA solution can subsequently be sequenced with a first and second sequencing primer using a next-generation sequencing technology.

In some embodiments, a sample processed using a system provided herein comprises genomic DNA. In some embodiments, samples comprising genomic DNA include a fragmentation step preceding step (a). In some embodiments, each ligation and amplification step can optionally comprise a subsequent purification step (e.g., sample purification between step (a) and step (b), sample purification between step (b) and step (c), and/or sample purification following step (c)). For example, the method of enriching samples comprising genomic DNA can comprise: (a) fragmentation of genomic DNA; (b) ligating a target nucleic acid comprising the known target nucleotide sequence with a universal oligonucleotide tail-adapter; (c) post-ligation sample purification; (d) amplifying a portion of the target nucleic acid and the amplification strand of the universal oligonucleotide tail-adapter with a first adapter primer and a first target-specific primer; (e) post-amplification sample purification; (f) amplifying a portion of the amplicon resulting from step (d) with a second adapter primer and a second target-specific primer; (g) post-amplification sample purification; and (h) transferring the purified DNA solution to a user. In some embodiments, steps of the methods may be performed within different vessels of a cartridge provided herein. In some embodiments, microfluidic channels and valves in the cartridge facilitate the transfer of reaction material/fluid from one vessel to another in the cartridge in an automated fashion. In The purified sample can subsequently be sequenced with a first and second sequencing primer using a next-generation sequencing technology.

In some embodiments, systems and methods provided herein may be used for processing nucleic acids as depicted in the exemplary workflow in FIG. 1. A nucleic acid sample 120 is provided. In some embodiments, the sample comprises RNA. In some embodiments, the sample comprises DNA (e.g., double-stranded complementary DNA (cDNA) and/or double-stranded genomic DNA (gDNA) 102). In some embodiments, the nucleic acid sample is subjected to a step 102 comprising nucleic acid end repair and/or dA tailing. In some embodiments, the nucleic acid sample is subjected to a step 104 comprising adapter ligation. In some embodiments, a universal oligonucleotide adapter 122 is ligated to one or more nucleic acids in the nucleic acid sample. In some embodiments, the ligation step comprises blunt-end ligation. In some embodiments, the ligation step comprises sticky-end ligation. In some embodiments, the ligation step comprises overhang ligation. In some embodiments, the ligation step comprises TA ligation. In some embodiments, the dA tailing step 102 is performed to generate an overhang in the nucleic acid sample that is complementary to an overhang in the universal oligonucleotide adapter (e.g., TA ligation). In some embodiments, a universal oligonucleotide adapter is ligated to both ends of one or more nucleic acids in the nucleic acid sample to generate a nucleic acid 124 flanked by universal oligonucleotide adapters. In some embodiments, an initial round of amplification is performed using an adapter primer 130 and a first target-specific primer 132. In some embodiments, the amplified sample is subjected to a second round of amplification using an adapter primer and a second target-specific primer 134. In some embodiments, the second target-specific primer is nested relative to the first target-specific primer. In some embodiments, the second target-specific primer comprises additional sequences 5′ to a hybridization sequence (e.g., common sequence) that may include barcode, index, adapter sequences, or sequencing primer sites. In some embodiments, the second target-specific primer is further contacted by an additional primer that hybridizes with the common sequence of the second target-specific primer, as depicted by 134. In some embodiments, the second round of amplification generates a nucleic acid 126 that is suitable for nucleic acid sequencing (e.g., next generation sequencing methods).

In some embodiments, systems and methods provided herein may be used for processing nucleic acids as described in PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and in PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which relating to nucleic acid library preparation are hereby incorporated by reference.

In some embodiments, a sample processed using a system provided herein comprises ribonucleic acid (RNA). In some embodiments, a system provided herein can be useful for processing RNA by a method comprising: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) amplifying a portion of the target nucleic acid and the amplification strand of the universal oligonucleotide tail-adapter with a first adapter primer and a first target-specific primer; (h) amplifying a portion of the amplicon resulting from step (g) with a second adapter primer and a second target-specific primer; and (i) transferring the cDNA solution to a user. In some embodiments, one or more steps of the methods may be performed within different vessels of a cartridge provided herein. In some embodiments, cDNA solution can subsequently be sequenced with a first and second sequencing primer using a next-generation sequencing technology.

In some embodiments, each ligation and amplification step can optionally comprise a subsequent sample purification step (e.g., sample purification step between step (f) and step (g), sample purification step between step (g) and step (h), and/or sample purification following step (h)). For example, the method of enriching samples comprising RNA can comprise: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) post-ligation sample purification; (h) amplifying a portion of the target nucleic acid and the amplification strand of the universal oligonucleotide tail-adapter with a first adapter primer and a first target-specific primer; (i) post-amplification sample purification; (j) amplifying a portion of the amplicon resulting from step (h) with a second adapter primer and a second target-specific primer; (k) post-amplification sample purification; and (l) transferring the purified cDNA solution to a user. In some embodiments, one or more steps of the methods may be performed within different vessels of a cartridge provided herein. The purified sample can subsequently be sequenced with a first and second sequencing primer using a next-generation sequencing technology.

In some embodiments, the systems provided herein may be configured to implement, e.g., in an automated fashion, a method of enriching nucleotide sequences that comprise a known target nucleotide sequence downstream from an adjacent region of unknown nucleotide sequence (e.g., nucleotide sequences comprising a 5′ region comprising an unknown sequence and a 3′ region comprising a known sequence). In some embodiments, the method comprises: (a) contacting a target nucleic acid molecule comprising the known target nucleotide sequence with an initial target-specific primer under hybridization conditions; (b) performing a template-dependent extension reaction that is primed by a hybridized initial target-specific primer and that uses the target nucleic acid molecule as a template; (c) contacting the product of step (b) with a population of tailed random primers under hybridization conditions; (d) performing a template-dependent extension reaction that is primed by a hybridized tailed random primer and that uses the portion of the target nucleic acid molecule downstream of the site of hybridization as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (e), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein.

In some embodiments, the systems provided herein may be configured to implement, e.g., in an automated fashion, a method of enriching nucleotide sequences that comprise a known target nucleotide sequence upstream from an adjacent region of unknown nucleotide sequence (e.g., nucleotide sequences comprising a 5′ region comprising a known sequence and a 3′ region comprising an unknown sequence). In some embodiments, the method comprises: (a) contacting a target nucleic acid molecule comprising the known target nucleotide sequence with a population of tailed random primers under hybridization conditions; (b) performing a template-dependent extension reaction that is primed by a hybridized tailed random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein. In some embodiments, the method further involves a step of contacting the sample with RNase after extension of the initial target-specific primer. In some embodiments, the tailed random primer can form a hair-pin loop structure. In some embodiments, the initial target-specific primer and the first target-specific primer are identical. In some embodiments, the tailed random primer further comprises a barcode portion comprising 6-12 random nucleotides between the 5′ nucleic acid sequence identical to a first sequencing primer and the 3′ nucleic acid sequence comprising 6-12 random nucleotides.

### Universal Oligonucleotide Tail Adapter

As used herein, the term “universal oligonucleotide tail-adapter” refers to a nucleic acid molecule comprised of two strands (a blocking strand and an amplification strand) and comprising a first ligatable duplex end and a second unpaired end. The blocking strand of the universal oligonucleotide tail-adapter comprises a 5′ duplex portion. The amplification strand comprises an unpaired 5′ portion, a 3′ duplex portion, a 3′ T overhang, and nucleic acid sequences identical to a first and second sequencing primer. The duplex portions of the blocking strand and the amplification strand are substantially complementary and form the first ligatable duplex end comprising a 3′ T overhang and the duplex portion is of sufficient length to remain in duplex form at the ligation temperature.

In some embodiments, the portion of the amplification strand that comprises a nucleic acid sequence identical to a first and second sequencing primer can be comprised, at least in part, by the 5′ unpaired portion of the amplification strand.

In some embodiments, the universal oligonucleotide tail-adapter can comprise a duplex portion and an unpaired portion, wherein the unpaired portion comprises only the 5′ portion of the amplification strand, i.e., the entirety of the blocking strand is a duplex portion.

In some embodiments, the universal oligonucleotide tail-adapter can have a “Y” shape, i.e., the unpaired portion can comprise portions of both the blocking strand and the amplification strand which are unpaired. The unpaired portion of the blocking strand can be shorter than, longer than, or equal in length to the unpaired portion of the amplification strand. In some embodiments, the unpaired portion of the blocking strand can be shorter than the unpaired portion of the amplification strand. Y shaped universal oligonucleotide tail-adapters have the advantage that the unpaired portion of the blocking strand will not be subject to 3′ extension during a PCR regimen.

In some embodiments, the blocking strand of the universal oligonucleotide tail-adapter can further comprise a 3′ unpaired portion which is not substantially complementary to the 5′ unpaired portion of the amplification strand; and wherein the 3′ unpaired portion of the blocking strand is not substantially complementary to or substantially identical to any of the primers. In some embodiments, the blocking strand of the universal oligonucleotide tail-adapter can further comprise a 3′ unpaired portion which will not specifically anneal to the 5′ unpaired portion of the amplification strand at the annealing temperature; and wherein the 3′ unpaired portion of the blocking strand will not specifically anneal to any of the primers or the complements thereof at the annealing temperature.

### First Amplification Step

As used herein, the term “first target-specific primer” refers to a single-stranded oligonucleotide comprising a nucleic acid sequence that can specifically anneal under suitable annealing conditions to a nucleic acid template that has a strand characteristic of a target nucleic acid.

In some embodiments, a primer (e.g., a target specific primer) can comprise a 5′ tag sequence portion. In some embodiments, multiple primers (e.g., all first-target specific primers) present in a reaction can comprise identical 5′ tag sequence portions. In some embodiments, in a multiplex PCR reaction, different primer species can interact with each other in an off-target manner, leading to primer extension and subsequently amplification by DNA polymerase. In such embodiments, these primer dimers tend to be short, and their efficient amplification can overtake the reaction and dominate resulting in poor amplification of desired target sequence. Accordingly, in some embodiments, the inclusion of a 5′ tag sequence in primers (e.g., on target specific primer(s)) may result in formation of primer dimers that contain the same complementary tails on both ends. In some embodiments, in subsequent amplification cycles, such primer dimers would denature into single-stranded DNA primer dimers, each comprising complementary sequences on their two ends which are introduced by the 5′ tag. In some embodiments, instead of primer annealing to these single stranded DNA primer dimers, an intra-molecular hairpin (a panhandle like structure) formation may occur due to the proximate accessibility of the complementary tags on the same primer dimer molecule instead of an inter-molecular interaction with new primers on separate molecules. Accordingly, in some embodiments, these primer dimers may be inefficiently amplified, such that primers are not exponentially consumed by the dimers for amplification; rather the tagged primers can remain in high and sufficient concentration for desired specific amplification of target sequences. In some embodiments, accumulation of primer dimers may be undesirable in the context of multiplex amplification because they compete for and consume other reagents in the reaction.

In some embodiments, a 5′ tag sequence can be a GC-rich sequence. In some embodiments, a 5′ tag sequence may comprise at least 50% GC content, at least 55% GC content, at least 60% GC content, at least 65% GC content, at least 70% GC content, at least 75% GC content, at least 80% GC content, or higher GC content. In some embodiments, a tag sequence may comprise at least 60% GC content. In some embodiments, a tag sequence may comprise at least 65% GC content.

As used herein, the term “first adapter primer” refers to a nucleic acid molecule comprising a nucleic acid sequence identical to a 5′ portion of the first sequencing primer. As the first tail-adapter primer is therefore identical to at least a portion of the sequence of the amplification strand (as opposed to complementary), it will not be able to specifically anneal to any portion of the universal oligonucleotide tail-adapter itself.

In the first PCR amplification cycle of the first amplification step, the first target-specific primer can specifically anneal to a template strand of any nucleic acid comprising the known target nucleotide sequence. Depending upon the orientation with which the first target-specific primer was designed, a sequence upstream or downstream of the known target nucleotide sequence will be synthesized as a strand complementary to the template strand. If, during the extension phase of PCR, the 5′ end of the template strand terminates in a ligated universal oligonucleotide tail-adapter, the 3′ end of the newly synthesized product strand will comprise sequence complementary to the first tail-adapter primer. In subsequent PCR amplification cycles, both the first target-specific primer and the first tail-adapter primer will be able to specifically anneal to the appropriate strands of the target nucleic acid sequence and the sequence between the known nucleotide target sequence and the universal oligonucleotide tail-adapter can be amplified (i.e., copied).

### Second Amplification Step

As used herein, the term “second target-specific primer” refers to a single-stranded oligonucleotide comprising a 3′ portion comprising a nucleic acid sequence that can specifically anneal to a portion of the known target nucleotide sequence comprised by the amplicon resulting from a preceding amplification step, and a 5′ portion comprising a nucleic acid sequence that is identical to a second sequencing primer. The second target-specific primer can be further contacted by an additional primer (e.g., a primer having 3′ sequencing adapter/index sequences) that hybridizes with the common sequence of the second target-specific primer. In some embodiments, the additional primer may comprise additional sequences 5′ to the hybridization sequence that may include barcode, index, adapter sequences, or sequencing primer sites. In some embodiments, the additional primer is a generic sequencing adapter/index primer. The second target-specific primer is nested with respect to the first target-specific primer. In some embodiments, the second target-specific primer is nested with respect to the first target-specific primer by at least 3 nucleotides, e.g., by 3 or more, 4 or more, 5 or more, 6 or more, 7 or more, 8 or more, 9 or more, 10 or more, or 15 or more nucleotides.

In some embodiments, all of the second target-specific primers present in a reaction comprise the same 5′ portion. In some embodiments, the 5′ portion of the second target-specific primers can serve to suppress primer dimers as described for the 5′ tag of the first target-specific primer described above herein.

In some embodiments, the first and second target-specific primers are substantially complementary to the same strand of the target nucleic acid. In some embodiments, the portions of the first and second target-specific primers that specifically anneal to the known target sequence can comprise a total of at least 20 unique bases of the known target nucleotide sequence, e.g., 20 or more unique bases, 25 or more unique bases, 30 or more unique bases, 35 or more unique bases, 40 or more unique bases, or 50 or more unique bases. In some embodiments, the portions of the first and second target-specific primers that specifically anneal to the known target sequence can comprise a total of at least 30 unique bases of the known target nucleotide sequence.

As used herein, the term “second adapter primer” refers to a nucleic acid molecule comprising a nucleic acid sequence identical to a portion of the first sequencing primer and is nested with respect to the first adapter primer. As the second tail-adapter primer is therefore identical to at least a portion of the sequence of the amplification strand (as opposed to complementary), it will not be able to specifically anneal to any portion of the universal oligonucleotide tail-adapter itself. In some embodiments, the second adapter primer is identical to the first sequencing primer.

The second adapter primer should be nested with respect to the first adapter primer, that is, the first adapter primer comprises a nucleic acid sequence identical to the amplification strand which is not comprised by the second adapter primer and which is located closer to the 5′ end of the amplification primer than any of the sequence identical to the amplification strand which is comprised by the second adapter primer. In some embodiments, the second adapter primer is nested by at least 3 nucleotides, e.g., by 3 nucleotides, by 4 nucleotides, by 5 nucleotides, by 6 nucleotides, by 7 nucleotides, by 8 nucleotides, by 9 nucleotides, by 10 nucleotides or more.

In some embodiments, the first adapter primer can comprise a nucleic acid sequence identical to about the 20 5′-most bases of the amplification strand of the universal oligonucleotide tail-adapter and the second adapter primer can comprise a nucleic acid sequence identical to about 30 bases of the amplification strand of the universal oligonucleotide tail-adapter, with a 5′ base which is at least 3 nucleotides 3′ of the 5′ terminus of the amplification strand.

In some embodiments, nested primer sets may be used. In some embodiments, the use of nested adapter primers eliminates the possibility of producing final amplicons that are amplifiable (e.g., during bridge PCR or emulsion PCR) but cannot be efficiently sequenced using certain techniques. In some embodiments, hemi-nested primer sets may be used.

### Sample Purification Step

In some embodiments, target nucleic acids and/or amplification products thereof can be isolated from enzymes, primers, or buffer components before and/or after any appropriate step of a method. Any suitable methods for isolating nucleic acids may be used. In some embodiments, the isolation can comprise Solid Phase Reversible Immobilization (SPRI) cleanup. Methods for SPRI cleanup are well known in the art, e.g., Agencourt AMPure XP-PCR Purification (Cat No. A63880, Beckman Coulter; Brea, Calif.). In some embodiments, enzymes can be inactivated by heat treatment.

In some embodiments, unhybridized primers can be removed from a nucleic acid preparation using appropriate methods (e.g., purification, digestion, etc.). In some embodiments, a nuclease (e.g., exonuclease I) is used to remove primer from a preparation. In some embodiments, such nucleases are heat inactivated subsequent to primer digestion. Once the nucleases are inactivated, a further set of primers may be added together with other appropriate components (e.g., enzymes, buffers) to perform a further amplification reaction.

### Sequencing

In some aspects, the technology described herein relates to methods of enriching nucleic acid samples for oligonucleotide sequencing. In some embodiments, the sequencing can be performed by a next-generation sequencing method. As used herein, “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ION Torrent); DNA nanoball sequencing (Complete Genomics); and technologies available from Pacific Biosciences, Intelligen Biosystems, and Oxford Nanopore Technologies. In some embodiments, the sequencing primers can comprise portions compatible with the selected next-generation sequencing method. Next-generation sequencing technologies and the constraints and design parameters of associated sequencing primers are well known in the art (see, e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R. Curr Opin Genet Dev 16:545-52 (2006); Strausberg, R. L., et al. Drug Disc Today 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; and 20070070349; which are incorporated by reference herein in their entireties).

In some embodiments, the sequencing step relies upon the use of a first and second sequencing primer. In some embodiments, the first and second sequencing primers are selected to be compatible with a next-generation sequencing method as described herein.

Methods of aligning sequencing reads to known sequence databases of genomic and/or cDNA sequences are well known in the art, and software is commercially available for this process. In some embodiments, reads (less the sequencing primer and/or adapter nucleotide sequence) which do not map, in their entirety, to wild-type sequence databases can be genomic rearrangements or large indel mutations. In some embodiments, reads (less the sequencing primer and/or adapter nucleotide sequence) comprising sequences which map to multiple locations in the genome can be genomic rearrangements.

### AMP Primers

In some embodiments, the four types of primers (first and second target-specific primers and first and second adapter primers) are designed such that they will specifically anneal to their complementary sequences at an annealing temperature of from about 61 to 72° C., e.g., from about 61 to 69° C., from about 63 to 69° C., from about 63 to 67° C., from about 64 to 66° C. In some embodiments, the four types of primers are designed such that they will specifically anneal to their complementary sequences at an annealing temperature of less than 72° C. In some embodiments, the four types of primers are designed such that they will specifically anneal to their complementary sequences at an annealing temperature of less than 70° C. In some embodiments, the four types of primers are designed such that they will specifically anneal to their complementary sequences at an annealing temperature of less than 68° C. In some embodiments, the four types of primers are designed such that they will specifically anneal to their complementary sequences at an annealing temperature of about 65° C. In some embodiments, systems provided herein are configured to alter vessel temperature (e.g., by cycling between different temperature ranges) to facilitate primer annealing.

In some embodiments, the portions of the target-specific primers that specifically anneal to the known target nucleotide sequence will anneal specifically at a temperature of about 61 to 72° C., e.g., from about 61 to 69° C., from about 63 to 69° C., from about 63 to 67° C., from about 64 to 66° C. In some embodiments, the portions of the target-specific primers that specifically anneal to the known target nucleotide sequence will anneal specifically at a temperature of about 65° C. in a PCR buffer.

In some embodiments, the primers and/or adapters described herein cannot comprise modified bases (e.g., the primers and/or adapters cannot comprise a blocking 3′ amine).

### Nucleic Acid Extension, Amplification, and PCR

In some embodiments, methods described herein comprise an extension regimen or step. In such embodiments, extension may proceed from one or more hybridized tailed random primers, using the nucleic acid molecules which the primers are hybridized to as templates. Extension steps are described herein. In some embodiments, one or more tailed random primers can hybridize to substantially all of the nucleic acids in a sample, many of which may not comprise a known target nucleotide sequence. Accordingly, in some embodiments, extension of random primers may occur due to hybridization with templates that do not comprise a known target nucleotide sequence.

In some embodiments, methods described herein may involve a polymerase chain reaction (PCR) amplification regimen, involving one or more amplification cycles.

Amplification steps of the methods described herein can each comprise a PCR amplification regimen, i.e., a set of polymerase chain reaction (PCR) amplification cycles. In some embodiments, systems provided herein are configured to alter vessel temperature (e.g., by cycling between different temperature ranges) to facilitate different PCR steps, e.g., melting, annealing, elongation, etc.

In some embodiments, system provided herein are configured to implement an amplification regimen in an automated fashion. As used herein, the term “amplification regimen” refers to a process of specifically amplifying (increasing the abundance of) a nucleic acid of interest. In some embodiments, exponential amplification occurs when products of a previous polymerase extension serve as templates for successive rounds of extension. In some embodiments, a PCR amplification regimen according to methods disclosed herein may comprise at least one, and in some cases at least 5 or more iterative cycles. In some embodiments, each iterative cycle comprises steps of: 1) strand separation (e.g., thermal denaturation); 2) oligonucleotide primer annealing to template molecules; and 3) nucleic acid polymerase extension of the annealed primers. In should be appreciated that any suitable conditions and times involved in each of these steps may be used. In some embodiments, conditions and times selected may depend on the length, sequence content, melting temperature, secondary structural features, or other factors relating to the nucleic acid template and/or primers used in the reaction. In some embodiments, an amplification regimen according to methods described herein is performed in a thermal cycler, many of which are commercially available.

In some embodiments, a nucleic acid extension reaction involves the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and are commercially available. One group of nucleic acid polymerases are thermostable, i.e., they retain function after being subjected to temperatures sufficient to denature annealed strands of complementary nucleic acids, e.g., 94° C., or sometimes higher. A non-limiting example of a protocol for amplification involves using a polymerase (e.g., Phoenix Taq, VeraSeq) under the following conditions: 98° C. for 30 s, followed by 14-22 cycles comprising melting at 98° C. for 10 s, followed by annealing at 68° C. for 30 s, followed by extension at 72° C. for 3 min, followed by holding of the reaction at 4° C. However, other appropriate reaction conditions may be used. In some embodiments, annealing/extension temperatures may be adjusted to account for differences in salt concentration (e.g., 3° C. higher to higher salt concentrations). In some embodiments, slowing the ramp rate (e.g., 1° C./s, 0.5° C./s, 0.28° C./s, 0.1° C./s or slower), for example, from 98° C. to 65° C., improves primer performance and coverage uniformity in highly multiplexed samples. In some embodiments, systems provided herein are configured to alter vessel temperature (e.g., by cycling between different temperature ranges, having controlled ramp up or down rates) to facilitate amplification.

In some embodiments, a nucleic acid polymerase is used under conditions in which the enzyme performs a template-dependent extension. In some embodiments, the nucleic acid polymerase is DNA polymerase I, Taq polymerase, Phoenix Taq polymerase, Phusion polymerase, T4 polymerase, T7 polymerase, Klenow fragment, Klenow exo-, phi29 polymerase, AMV reverse transcriptase, M-MuLV reverse transcriptase, HIV-1 reverse transcriptase, VeraSeq ULtra polymerase, VeraSeq HF 2.0 polymerase, EnzScript, or another appropriate polymerase. In some embodiments, a nucleic acid polymerase is not a reverse transcriptase. In some embodiments, a nucleic acid polymerase acts on a DNA template. In some embodiments, the nucleic acid polymerase acts on an RNA template. In some embodiments, an extension reaction involves reverse transcription performed on an RNA to produce a complementary DNA molecule (RNA-dependent DNA polymerase activity). In some embodiments, a reverse transcriptase is a mouse moloney murine leukemia virus (M-MLV) polymerase, AMV reverse transcriptase, RSV reverse transcriptase, HIV-1 reverse transcriptase, HIV-2 reverse transcriptase, or another appropriate reverse transcriptase.

In some embodiments, a nucleic acid amplification reaction involves cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. In some embodiments, strand separation according to methods described herein is achieved by heating the nucleic acid sample above its melting temperature (Tₘ). In some embodiments, for a sample containing nucleic acid molecules in a reaction preparation suitable for a nucleic acid polymerase, heating to 94° C. is sufficient to achieve strand separation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), and a carrier (e.g., 0.01 to 0.5% BSA). A non-limiting example of a suitable buffer comprises 50 mM KCl, 10 mM Tris-HCl (pH 8.8 at 25° C.), 0.5 to 3 mM MgCl₂, and 0.1% BSA.

In some embodiments, a nucleic acid amplification involves annealing primers to nucleic acid templates having a strands characteristic of a target nucleic acid. In some embodiments, a strand of a target nucleic acid can serve as a template nucleic acid.

As used herein, the term “anneal” refers to the formation of one or more complementary base pairs between two nucleic acids. In some embodiments, annealing involves two complementary or substantially complementary nucleic acid strands hybridizing together. In some embodiments, in the context of an extension reaction, annealing involves the hybridization of primer to a template such that a primer extension substrate for a template-dependent polymerase enzyme is formed. In some embodiments, conditions for annealing (e.g., between a primer and nucleic acid template) may vary based of the length and sequence of a primer. In some embodiments, conditions for annealing are based upon a Tₘ (e.g., a calculated Tₘ) of a primer. In some embodiments, an annealing step of an extension regimen involves reducing the temperature following a strand separation step to a temperature based on the Tₘ (e.g., a calculated Tₘ) for a primer, for a time sufficient to permit such annealing. In some embodiments, a Tₘ can be determined using any of a number of algorithms (e.g., OLIGO™ (Molecular Biology Insights Inc. Colorado) primer design software and VENTRO NTI™ (Invitrogen, Inc. California) primer design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html)). In some embodiments, the Tₘ of a primer can be calculated using the following formula, which is used by NetPrimer software and is described in more detail in Frieir, et al. PNAS 1986 83:9373-9377 which is incorporated by reference herein in its entirety.

Tₘ=ΔH/(ΔS+R*ln(C/4))+16.6 log([K⁺]/(1+0.7[K⁺]))−273.15

wherein: ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ(e.g., 6° C. below, 8° C. below, 10° C. below or lower). In some embodiments, the closer an annealing temperature is to the Tₘ, the more specific is the annealing. In some embodiments, the time used for primer annealing during an extension reaction (e.g., within the context of a PCR amplification regimen) is determined based, at least in part, upon the volume of the reaction (e.g., with larger volumes involving longer times). In some embodiments, the time used for primer annealing during an extension reaction (e.g., within the context of a PCR amplification regimen) is determined based, at least in part, upon primer and template concentrations (e.g., with higher relative concentrations of primer to template involving less time than lower relative concentrations). In some embodiments, depending upon volume and relative primer/template concentration, primer annealing steps in an extension reaction (e.g., within the context of an amplification regimen) can be in the range of 1 second to 5 minutes, 10 seconds to 2 minutes, or 30 seconds to 2 minutes. As used herein, “substantially anneal” refers to an extent to which complementary base pairs form between two nucleic acids that, when used in the context of a PCR amplification regimen, is sufficient to produce a detectable level of a specifically amplified product.

As used herein, the term “polymerase extension” refers to template-dependent addition of at least one complementary nucleotide, by a nucleic acid polymerase, to the 3′ end of a primer that is annealed to a nucleic acid template. In some embodiments, polymerase extension adds more than one nucleotide, e.g., up to and including nucleotides corresponding to the full length of the template. In some embodiments, conditions for polymerase extension are based, at least in part, on the identity of the polymerase used. In some embodiments, the temperature used for polymerase extension is based upon the known activity properties of the enzyme. In some embodiments, in which annealing temperatures are below the optimal temperatures for the enzyme, it may be acceptable to use a lower extension temperature. In some embodiments, enzymes may retain at least partial activity below their optimal extension temperatures. In some embodiments, a polymerase extension (e.g., performed with thermostable polymerases such as Taq polymerase and variants thereof) is performed at 65° C. to 75° C. or 68° C. to 72° C. In some embodiments, methods provided herein involve polymerase extension of primers that are annealed to nucleic acid templates at each cycle of a PCR amplification regimen. In some embodiments, a polymerase extension is performed using a polymerase that has relatively strong strand displacement activity. In some embodiments, polymerases having strong strand displacement are useful for preparing nucleic acids for purposes of detecting fusions (e.g., 5′ fusions).

In some embodiments, primer extension is performed under conditions that permit the extension of annealed oligonucleotide primers. As used herein, the term “conditions that permit the extension of an annealed oligonucleotide such that extension products are generated” refers to the set of conditions (e.g., temperature, salt and co-factor concentrations, pH, and enzyme concentration) under which a nucleic acid polymerase catalyzes primer extension. In some embodiments, such conditions are based, at least in part, on the nucleic acid polymerase being used. In some embodiments, a polymerase may perform a primer extension reaction in a suitable reaction preparation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), a carrier (e.g., 0.01 to 0.5% BSA), and one or more NTPs (e.g., 10 to 200 μM of each of dATP, dTTP, dCTP, and dGTP). A non-limiting set of conditions is 50 mM KCl, 10 mM Tris-HCl (pH 8.8 at 25° C.), 0.5 to 3 mM MgCl₂, 200 μM each dNTP, and 0.1% BSA at 72° C., under which a polymerase (e.g., Taq polymerase) catalyzes primer extension. In some embodiments, conditions for initiation and extension may include the presence of one, two, three or four different deoxyribonucleoside triphosphates (e.g., selected from dATP, dTTP, dCTP, and dGTP) and a polymerization-inducing agent such as DNA polymerase or reverse transcriptase, in a suitable buffer. In some embodiments, a “buffer” may include solvents (e.g., aqueous solvents) plus appropriate cofactors and reagents which affect pH, ionic strength, etc.

In some embodiments, systems provided herein are configured to implement in an automated fashion multiple nucleic acid amplification cycles. In some embodiments, nucleic acid amplification involve up to 5, up to 10, up to 20, up to 30, up to 40 or more rounds (cycles) of amplification. In some embodiments, nucleic acid amplification may comprise a set of cycles of a PCR amplification regimen from 5 cycles to 20 cycles in length. In some embodiments, an amplification step may comprise a set of cycles of a PCR amplification regimen from 10 cycles to 20 cycles in length. In some embodiments, each amplification step can comprise a set of cycles of a PCR amplification regimen from 12 cycles to 16 cycles in length. In some embodiments, an annealing temperature can be less than 70° C. In some embodiments, an annealing temperature can be less than 72° C. In some embodiments, an annealing temperature can be about 65° C. In some embodiments, an annealing temperature can be from about 61 to about 72° C.

In various embodiments, methods and compositions described herein relate to performing a PCR amplification regimen with one or more of the types of primers described herein. As used herein, “primer” refers to an oligonucleotide capable of specifically annealing to a nucleic acid template and providing a 3′ end that serves as a substrate for a template-dependent polymerase to produce an extension product which is complementary to the template. In some embodiments, a primer is single-stranded, such that the primer and its complement can anneal to form two strands. Primers according to methods and compositions described herein may comprise a hybridization sequence (e.g., a sequence that anneals with a nucleic acid template) that is less than or equal to 300 nucleotides in length, e.g., less than or equal to 300, or 250, or 200, or 150, or 100, or 90, or 80, or 70, or 60, or 50, or 40, or 30 or fewer, or 20 or fewer, or 15 or fewer, but at least 6 nucleotides in length. In some embodiments, a hybridization sequence of a primer may be 6 to 50 nucleotides in length, 6 to 35 nucleotides in length, 6 to 20 nucleotides in length, 10 to 25 nucleotides in length.

Any suitable method may be used for synthesizing oligonucleotides and primers. In some embodiments, commercial sources offer oligonucleotide synthesis services suitable for providing primers for use in methods and compositions described herein (e.g., INVITROGEN™ Custom DNA Oligos (Life Technologies, Grand Island, N.Y.) or custom DNA Oligos from Integrated DNA Technologies (Coralville, Iowa)).

### DNA Shearing/Fragmentation

Nucleic acids used herein (e.g., prior to sequencing) can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a nucleic acid can be mechanically sheared by sonication. In some embodiments, systems provided here may have one or more vessels, e.g., within a cassette that is fitted within a cartridge, in which nucleic acids are sheared, e.g., mechanically or enzymatically.

In some embodiments, a target nucleic acid is not sheared or digested. In some embodiments, nucleic acid products of preparative steps (e.g., extension products, amplification products) are not sheared or enzymatically digested.

In some embodiments, when a target nucleic acid is RNA, the sample can be subjected to a reverse transcriptase regimen to generate a DNA template and the DNA template can then be sheared. In some embodiments, target RNA can be sheared before performing a reverse transcriptase regimen. In some embodiments, a sample comprising target RNA can be used in methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers.

### Target Nucleic Acid

As used herein, the term “target nucleic acid” refers to a nucleic acid molecule of interest (e.g., a nucleic acid to be analyzed). In some embodiments, a target nucleic acid comprises both a target nucleotide sequence (e.g., a known or predetermined nucleotide sequence) and an adjacent nucleotide sequence which is to be determined (which may be referred to as an unknown sequence). A target nucleic acid can be of any appropriate length. In some embodiments, a target nucleic acid is double-stranded. In some embodiments, the target nucleic acid is DNA. In some embodiments, the target nucleic acid is genomic or chromosomal DNA (gDNA). In some embodiments, the target nucleic acid can be complementary DNA (cDNA). In some embodiments, the target nucleic acid is single-stranded. In some embodiments, the target nucleic acid can be RNA (e.g., mRNA, rRNA, tRNA, long non-coding RNA, microRNA).

In some embodiments, the target nucleic acid can be comprised by genomic DNA. In some embodiments, the target nucleic acid can be comprised by ribonucleic acid (RNA), e.g., mRNA. In some embodiments, the target nucleic acid can be comprised by cDNA. Many of the sequencing methods suitable for use in the methods described herein provide sequencing runs with optimal read lengths of tens to hundreds of nucleotide bases (e.g., Ion Torrent technology can produce read lengths of 200-400 bp). Target nucleic acids comprised, for example, by genomic DNA or mRNA, can be comprised by nucleic acid molecules which are substantially longer than this optimal read length. In order for the amplified nucleic acid portion resulting from the second amplification step to be of a suitable length for use in a particular sequencing technology, the average distance between the known target nucleotide sequence and an end of the target nucleic acid to which the universal oligonucleotide tail-adapter can be ligated should be as close to the optimal read length of the selected technology as possible. For example, if the optimal read-length of a given sequencing technology is 200 bp, then the nucleic acid molecules amplified in accordance with the methods described herein should have an average length of about 400 bp or less. Target nucleic acids comprised by, e.g., genomic DNA or mRNA, can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a target nucleic acid comprised by genomic DNA can be mechanically sheared by sonication.

In some embodiments, when the target nucleic acid is comprised by RNA, the sample can be subjected to a reverse transcriptase regimen to generate a DNA template and the DNA template can then be sheared. In some embodiments, target RNA can be sheared before performing the reverse transcriptase regimen. In some embodiments, a sample comprising target RNA can be used in the methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers; and by subjecting the nucleic acid to end-repair, phosphorylation, and adenylation.

In some embodiments, the known target nucleotide sequence can be comprised by a gene rearrangement. The methods described herein are suited for determining the presence and/or identity of a gene rearrangement as the identity of only one half of the gene rearrangement must be previously known (i.e., the half of the gene rearrangement which is to be targeted by the gene-specific primers). In some embodiments, the gene rearrangement can comprise an oncogene. In some embodiments, the gene rearrangement can comprise a fusion oncogene.

As used herein, the term “known target nucleotide sequence” refers to a portion of a target nucleic acid for which the sequence (e.g., the identity and order of the nucleotide bases of the nucleic acid) is known. For example, in some embodiments, a known target nucleotide sequence is a nucleotide sequence of a nucleic acid that is known or that has been determined in advance of an interrogation of an adjacent unknown sequence of the nucleic acid. A known target nucleotide sequence can be of any appropriate length.

In some embodiments, a target nucleotide sequence (e.g., a known target nucleotide sequence) has a length of 10 or more nucleotides, 30 or more nucleotides, 40 or more nucleotides, 50 or more nucleotides, 100 or more nucleotides, 200 or more nucleotides, 300 or more nucleotides, 400 or more nucleotides, 500 or more nucleotides. In some embodiments, a target nucleotide sequence (e.g., a known target nucleotide sequence) has a length in the range of 10 to 100 nucleotides, 10 to 500 nucleotides, 10 to 1000 nucleotides, 100 to 500 nucleotides, 100 to 1000 nucleotides, 500 to 1000 nucleotides, 500 to 5000 nucleotides.

In some embodiments, methods are provided herein for determining sequences of contiguous (or adjacent) portions of a nucleic acid. As used herein, the term “nucleotide sequence contiguous to” refers to a nucleotide sequence of a nucleic acid molecule (e.g., a target nucleic acid) that is immediately upstream or downstream of another nucleotide sequence (e.g., a known nucleotide sequence). In some embodiments, a nucleotide sequence contiguous to a known target nucleotide sequence may be of any appropriate length. In some embodiments, a nucleotide sequence contiguous to a known target nucleotide sequence comprises 1 kb or less of nucleotide sequence, e.g., 1 kb or less of nucleotide sequence, 750 bp or less of nucleotide sequence, 500 bp or less of nucleotide sequence, 400 bp or less of nucleotide sequence, 300 bp or less of nucleotide sequence, 200 bp or less of nucleotide sequence, 100 bp or less of nucleotide sequence. In some embodiments, in which a sample comprises different target nucleic acids comprising a known target nucleotide sequence (e.g., a cell in which a known target nucleotide sequence occurs multiple times in its genome, or on separate, non-identical chromosomes), there may be multiple sequences which comprise “a nucleotide sequence contiguous to” the known target nucleotide sequence. As used herein, the term “determining a (or the) nucleotide sequence,” refers to determining the identity and relative positions of the nucleotide bases of a nucleic acid.

In some embodiments, a known target nucleic acid can contain a fusion sequence resulting from a gene rearrangement. In some embodiments, methods described herein are suited for determining the presence and/or identity of a gene rearrangement. In some embodiments, the identity of one portion of a gene rearrangement is previously known (e.g., the portion of a gene rearrangement that is to be targeted by the gene-specific primers) and the sequence of the other portion may be determined using methods disclosed herein. In some embodiments, a gene rearrangement can involve an oncogene. In some embodiments, a gene rearrangement can comprise a fusion oncogene.

### Samples

In some embodiments, a target nucleic acid is present in or obtained from an appropriate sample (e.g., a food sample, environmental sample, biological sample e.g., blood sample, etc.). In some embodiments, the target nucleic acid is a biological sample obtained from a subject. In some embodiments a sample can be a diagnostic sample obtained from a subject. In some embodiments, a sample can further comprise proteins, cells, fluids, biological fluids, preservatives, and/or other substances. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample can be obtained from a subject in need of treatment for a disease associated with a genetic alteration, e.g., cancer or a hereditary disease. In some embodiments, a known target sequence is present in a disease-associated gene.

In some embodiments, a sample is obtained from a subject in need of treatment for cancer. In some embodiments, the sample comprises a population of tumor cells, e.g., at least one tumor cell. In some embodiments, the sample comprises a tumor biopsy, including but not limited to, untreated biopsy tissue or treated biopsy tissue (e.g., formalin-fixed and/or paraffin-embedded biopsy tissue).

In some embodiments, the sample is freshly collected. In some embodiments, the sample is stored prior to being used in methods and compositions described herein. In some embodiments, the sample is an untreated sample. As used herein, “untreated sample” refers to a biological sample that has not had any prior sample pre-treatment except for dilution and/or suspension in a solution. In some embodiments, a sample is obtained from a subject and preserved or processed prior to being utilized in methods and compositions described herein. By way of non-limiting example, a sample can be embedded in paraffin wax, refrigerated, or frozen. A frozen sample can be thawed before determining the presence of a nucleic acid according to methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g., anti-coagulant or nuclease inhibitor) and any combination thereof. In some embodiments, a sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in methods and compositions described herein. Suitable methods and processes for processing, preservation, or treatment of samples for nucleic acid analysis may be used in the method disclosed herein. In some embodiments, a sample can be a clarified fluid sample. In some embodiments, a sample can be clarified by low-speed centrifugation (e.g., 3,000×g or less) and collection of the supernatant comprising the clarified fluid sample.

In some embodiments, a nucleic acid present in a sample can be isolated, enriched, or purified prior to being utilized in methods and compositions described herein. Suitable methods of isolating, enriching, or purifying nucleic acids from a sample may be used. For example, kits for isolation of genomic DNA from various sample types are commercially available (e.g., Catalog Nos. 51104, 51304, 56504, and 56404; Qiagen; Germantown, Md.). In some embodiments, methods described herein relate to methods of enriching for target nucleic acids, e.g., prior to a sequencing of the target nucleic acids. In some embodiments, a sequence of one end of the target nucleic acid to be enriched is not known prior to sequencing. In some embodiments, methods described herein relate to methods of enriching specific nucleotide sequences prior to determining the nucleotide sequence using a next-generation sequencing technology. In some embodiments, methods of enriching specific nucleotide sequences do not comprise hybridization enrichment.

### Target Genes (ALK, ROS1, RET) and Therapeutic Applications

In some embodiments of methods described herein, a determination of the sequence contiguous to a known oligonucleotide target sequence can provide information relevant to treatment of disease. Thus, in some embodiments, methods disclosed herein can be used to aid in treating disease. In some embodiments, a sample can be from a subject in need of treatment for a disease associated with a genetic alteration. In some embodiments, a known target sequence is a sequence of a disease-associated gene, e.g., an oncogene. In some embodiments, a sequence contiguous to a known oligonucleotide target sequence and/or the known oligonucleotide target sequence can comprise a mutation or genetic abnormality which is disease-associated, e.g., a SNP, an insertion, a deletion, and/or a gene rearrangement. In some embodiments, a sequence contiguous to a known target sequence and/or a known target sequence present in a sample comprised sequence of a gene rearrangement product. In some embodiments, a gene rearrangement can be an oncogene, e.g., a fusion oncogene.

Certain treatments for cancer are particularly effective against tumors comprising certain oncogenes, e.g., a treatment agent which targets the action or expression of a given fusion oncogene can be effective against tumors comprising that fusion oncogene but not against tumors lacking the fusion oncogene. Methods described herein can facilitate a determination of specific sequences that reveal oncogene status (e.g., mutations, SNPs, and/or rearrangements). In some embodiments, methods described herein can further allow the determination of specific sequences when the sequence of a flanking region is known, e.g., methods described herein can determine the presence and identity of gene rearrangements involving known genes (e.g., oncogenes) in which the precise location and/or rearrangement partner are not known before methods described herein are performed.

In some embodiments, a subject is in need of treatment for lung cancer. In some embodiments, e.g., when the sample is obtained from a subject in need of treatment for lung cancer, the known target sequence can comprise a sequence from a gene selected from the group of ALK, ROS1, and RET. Accordingly, in some embodiments, gene rearrangements result in fusions involving the ALK, ROS1, or RET. Non-limiting examples of gene arrangements involving ALK, ROS1, or RET are described in, e.g., Soda et al. Nature 2007 448561-6: Rikova et al. Cell 2007 131:1190-1203; Kohno et al. Nature Medicine 2012 18:375-7; Takouchi et al. Nature Medicine 2012 18:378-81; which are incorporated by reference herein in their entireties. However, it should be appreciated that the precise location of a gene rearrangement and the identity of the second gene involved in the rearrangement may not be known in advance. Accordingly, in methods described herein, the presence and identity of such rearrangements can be detected without having to know the location of the rearrangement or the identity of the second gene involved in the gene rearrangement.

In some embodiments, the known target sequence can comprise sequence from a gene selected from the group of: ALK, ROS1, and RET.

In some embodiments, the presence of a gene rearrangement of ALK in a sample obtained from a tumor in a subject can indicate that the tumor is susceptible to treatment with a treatment selected from the group consisting of: an ALK inhibitor; crizotinib (PF-02341066); AP26113; LDK378; 3-39; AF802; IPI-504; ΔSP3026; AP-26113; X-396; GSK-1838705A; CH5424802; diamino and aminopyrimidine inhibitors of ALK kinase activity such as NVP-TAE684 and PF-02341066 (see, e.g., Galkin et al., Proc Natl Acad Sci USA, 2007, 104:270-275; Zou et al., Cancer Res, 2007, 67:4408-4417; Hallberg and Palmer F1000 Med Reports 2011 3:21; Sakamoto et al., Cancer Cell 2011 19:679-690; and molecules disclosed in WO 04/079326). All of the foregoing references are incorporated by reference herein in their entireties. An ALK inhibitor can include any agent that reduces the expression and/or kinase activity of ALK or a portion thereof, including, e.g., oligonucleotides, small molecules, and/or peptides that reduce the expression and/or activity of ALK or a portion thereof. As used herein “anaplastic lymphoma kinase” or “ALK” refers to a transmembrane tyROS line kinase typically involved in neuronal regulation in the wildtype form. The nucleotide sequence of the ALK gene and mRNA are known for a number of species, including human (e.g., as annotated under NCBI Gene ID: 238).

In some embodiments, the presence of a gene rearrangement of ROS1 in a sample obtained from a tumor in a subject can indicate that the tumor is susceptible to treatment with a treatment selected from the group consisting of: a ROS1 inhibitor and an ALK inhibitor as described herein above (e.g., crizotinib). A ROS1 inhibitor can include any agent that reduces the expression and/or kinase activity of ROS1 or a portion thereof, including, e.g., oligonucleotides, small molecules, and/or peptides that reduce the expression and/or activity of ROS1 or a portion thereof. As used herein “c-ros oncogene 1” or “ROS1” (also referred to in the art as ros-1) refers to a transmembrane tyrosine kinase of the sevenless subfamily and which interacts with PTPN6. Nucleotide sequences of the ROS1 gene and mRNA are known for a number of species, including human (e.g., as annotated under NCBI Gene ID: 6098).

In some embodiments, the presence of a gene rearrangement of RET in a sample obtained from a tumor in a subject can indicate that the tumor is susceptible to treatment with a treatment selected from the group consisting of: a RET inhibitor; DP-2490, DP-3636, SU5416; BAY 43-9006, BAY 73-4506 (regorafenib), ZD6474, NVP-ΔST487, sorafenib, RPI-1, XL184, vandetanib, sunitinib, imatinib, pazopanib, axitinib, motesanib, gefitinib, and withaferin A (see, e.g., Samadi et al., Surgery 2010 148:1228-36; Cuccuru et al., JNCI 2004 13:1006-1014; Akeno-Stuart et al., Cancer Research 2007 67:6956; Grazma et al., J Clin Oncol 2010 28:15s 5559; Mologni et al., J Mol Endocrinol 2006 37:199-212; Calmomagno et al., Journal NCI 2006 98:326-334; Mologni, Curr Med Chem 2011 18:162-175; and the compounds disclosed in WO 06/034833; US Patent Publication 2011/0201598 and U.S. Pat. No. 8,067,434). All of the foregoing references are incorporated by reference herein in their entireties. A RET inhibitor can include any agent that reduces the expression and/or kinase activity of RET or a portion thereof, including, e.g., oligonucleotides, small molecules, and/or peptides that reduce the expression and/or activity of RET or a portion thereof. As used herein, “rearranged during transfection” or “RET” refers to a receptor tyrosine kinase of the cadherin superfamily which is involved in neural crest development and recognizes glial cell line-derived neurotrophic factor family signaling molecules. Nucleotide sequences of the RET gene and mRNA are known for a number of species, including human (e.g., as annotated under NCBI Gene ID: 5979).

Further non-limiting examples of applications of methods described herein include detection of hematological malignancy markers and panels thereof (e.g., including those to detect genomic rearrangements in lymphomas and leukemias), detection of sarcoma-related genomic rearrangements and panels thereof; and detection of IGH/TCR gene rearrangements and panels thereof for lymphoma testing.

In some embodiments, methods described herein relate to treating a subject having or diagnosed as having, e.g., cancer with a treatment for cancer. Subjects having cancer can be identified by a physician using current methods of diagnosing cancer. For example, symptoms and/or complications of lung cancer which characterize these conditions and aid in diagnosis are well known in the art and include but are not limited to, weak breathing, swollen lymph nodes above the collarbone, abnormal sounds in the lungs, dullness when the chest is tapped, and chest pain. Tests that may aid in a diagnosis of, e.g., lung cancer include, but are not limited to, x-rays, blood tests for high levels of certain substances (e.g., calcium), CT scans, and tumor biopsy. A family history of lung cancer, or exposure to risk factors for lung cancer (e.g., smoking or exposure to smoke and/or air pollution) can also aid in determining if a subject is likely to have lung cancer or in making a diagnosis of lung cancer.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non-Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chronic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibROS larcoma, and osteosarcoma; pancreatic cancer; skin cancer including melanoma, stromal cells, germ cells and mesenchymal cells; pROS ltate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer.

### Multiplex Methods

Methods described herein can be employed in a multiplex format. In embodiments of methods described herein, multiplex applications can include determining the nucleotide sequence contiguous to one or more known target nucleotide sequences. As used herein, “multiplex amplification” refers to a process that involves simultaneous amplification of more than one target nucleic acid in one or more reaction vessels. In some embodiments, methods involve subsequent determination of the sequence of the multiplex amplification products using one or more sets of primers. Multiplex can refer to the detection of between about 2-1,000 different target sequences in a single reaction. As used herein, multiplex refers to the detection of any range between 2-1,000, e.g., between 5-500, 25-1,000, or 10-100 different target sequences in a single reaction, etc. The term “multiplex” as applied to PCR implies that there are primers specific for at least two different target sequences in the same PCR reaction.

In some embodiments, target nucleic acids in a sample, or separate portions of a sample, can be amplified with a plurality of primers (e.g., a plurality of first and second target-specific primers). In some embodiments, the plurality of primers (e.g., a plurality of first and second target-specific primers) can be present in a single reaction mixture, e.g., multiple amplification products can be produced in the same reaction mixture. In some embodiments, the plurality of primers (e.g., a plurality of sets of first and second target-specific primers) can specifically anneal to known target sequences comprised by separate genes. In some embodiments, at least two sets of primers (e.g., at least two sets of first and second target-specific primers) can specifically anneal to different portions of a known target sequence. In some embodiments, at least two sets of primers (e.g., at least two sets of first and second target-specific primers) can specifically anneal to different portions of a known target sequence comprised by a single gene. In some embodiments, at least two sets of primers (e.g., at least two sets of first and second target-specific primers) can specifically anneal to different exons of a gene comprising a known target sequence. In some embodiments, the plurality of primers (e.g., first target-specific primers) can comprise identical 5′ tag sequence portions.

In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g., from different tissues and/or different subjects. In such embodiments, primers (e.g., tailed random primers) can further comprise a barcode portion. In some embodiments, a primer (e.g., a tailed random primer) with a unique barcode portion can be added to each sample and ligated to the nucleic acids therein; the samples can subsequently be pooled. In such embodiments, each resulting sequencing read of an amplification product will comprise a barcode that identifies the sample containing the template nucleic acid from which the amplification product is derived.

### Molecular Barcodes

In some embodiments, primers may contain additional sequences such as an identifier sequence (e.g., a barcode, an index), sequencing primer hybridization sequences (e.g., Rdl), and adapter sequences. In some embodiments the adapter sequences are sequences used with a next generation sequencing system. In some embodiments, the adapter sequences are P5 and P7 sequences for Illumina-based sequencing technology. In some embodiments, the adapter sequence are P1 and A compatible with Ion Torrent sequencing technology.

In some embodiments, as used herein, “molecular barcode,” “molecular barcode tag,” and “index” may be used interchangeably, and generally refer to a nucleotide sequence of a nucleic acid that is useful as an identifier, such as, for example, a source identifier, location identifier, date or time identifier (e.g., date or time of sampling or processing), or other identifier of the nucleic acid. In some embodiments, such molecular barcode or index sequences are useful for identifying different aspects of a nucleic acid that is present in a population of nucleic acids. In some embodiments, molecular barcode or index sequences may provide a source or location identifier for a target nucleic acid. For example, a molecular barcode or index sequence may serve to identify a patient from whom a nucleic acid is obtained. In some embodiments, molecular barcode or index sequences enable sequencing of multiple different samples on a single reaction (e.g., performed in a single flow cell). In some embodiments, an index sequence can be used to orientate a sequence imager for purposes of detecting individual sequencing reactions. In some embodiments, a molecular barcode or index sequence may be 2 to 25 nucleotides in length, 2 to 15 nucleotides in length, 2 to 10 nucleotides in length, 2 to 6 nucleotides in length. In some embodiments, a barcode or index comprise at least 2, 3, 4, 5, 6, 7, 8, 9, 10, 11, 12, 13, 14, 15, 16, 17, 18, 19, 20, 21, 22, 23, 24, or at least 25 nucleotides.

In some embodiments, when a population of tailed random primers is used in accordance with methods described herein, multiple distinguishable amplification products can be present after amplification. In some embodiments, because tailed random primers hybridize at various positions throughout nucleic acid molecules of a sample, a set of target-specific primers can hybridize (and amplify) the extension products created by more than 1 hybridization event, e.g., one tailed random primer may hybridize at a first distance (e.g., 100 nucleotides) from a target-specific primer hybridization site, and another tailed random primer can hybridize at a second distance (e.g., 200 nucleotides) from a target-specific primer hybridization site, thereby resulting in two amplification products (e.g., a first amplification product comprising about 100 bp and a second amplification product comprising about 200 bp). In some embodiments, these multiple amplification products can each be sequenced using next generation sequencing technology. In some embodiments, sequencing of these multiple amplification products is advantageous because it provides multiple overlapping sequence reads that can be compared with one another to detect sequence errors introduced during amplification or sequencing processes. In some embodiments, individual amplification products can be aligned and where they differ in the sequence present at a particular base, an artifact or error of PCR and/or sequencing may be present.

### Computer and Control Equipment

The systems provided herein include several components, including sensors, environmental control systems (e.g., heaters, fans), robotics (e.g., an XY positioner), etc. which may operate together at the direction of a computer, processor, microcontroller or other controller. The components may include, for example, an XY positioner, a liquid handling devices, microfluidic pumps, linear actuators, valve drivers, a door operation system, an optics assembly, barcode scanners, imaging or detection system, touchscreen interface, etc.

In some cases, operations such as controlling operations of a systems and/or components provided therein or interfacing therewith may be implemented using hardware, software or a combination thereof. When implemented in software, the software code can be executed on any suitable processor or collection of processors, whether provided in a single component or distributed among multiple components. Such processors may be implemented as integrated circuits, with one or more processors in an integrated circuit component. A processor may be implemented using circuitry in any suitable format.

A computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable, mobile or fixed electronic device, including the system itself.

In some cases, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. In other examples, a computer may receive input information through speech recognition or in other audible format, through visible gestures, through haptic input (e.g., including vibrations, tactile and/or other forces), or any combination thereof.

One or more computers may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Such software may be written using any of a number of suitable programming languages and/or programming or scripting tools, and may be compiled as executable machine language code or intermediate code that is executed on a framework or virtual machine.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

In some embodiments, information related to the operation of the system (e.g., temperature, imaging or optical information, fluorescent signals, component positions (e.g., heated lid position, rotary valve position), liquid handling status, barcode status, bay access door position or any combination thereof) can be obtained from one or more sensors or readers associated with the system (e.g., located within the system), and can be stored in computer-readable media to provide information about conditions during a process (e.g., an automated library preparation process). In some embodiments, the readable media comprises a database. In some embodiments, said database contains data from a single system (e.g., from one or more bays). In some embodiments, said database contains data from a plurality of systems. In some embodiments, data is stored in a manner that makes it tamper-proof. In some embodiments, all data generated by the system is stored. In some embodiments, a subset of data is stored.

### EXAMPLES

The following examples are intended to illustrate certain embodiments described herein, including certain aspects of the present invention, but do not exemplify the full scope of the invention.

### Example 1

The following example demonstrates the operation of a channel system such that a sample fluid is transferred to a vessel of the channel system.

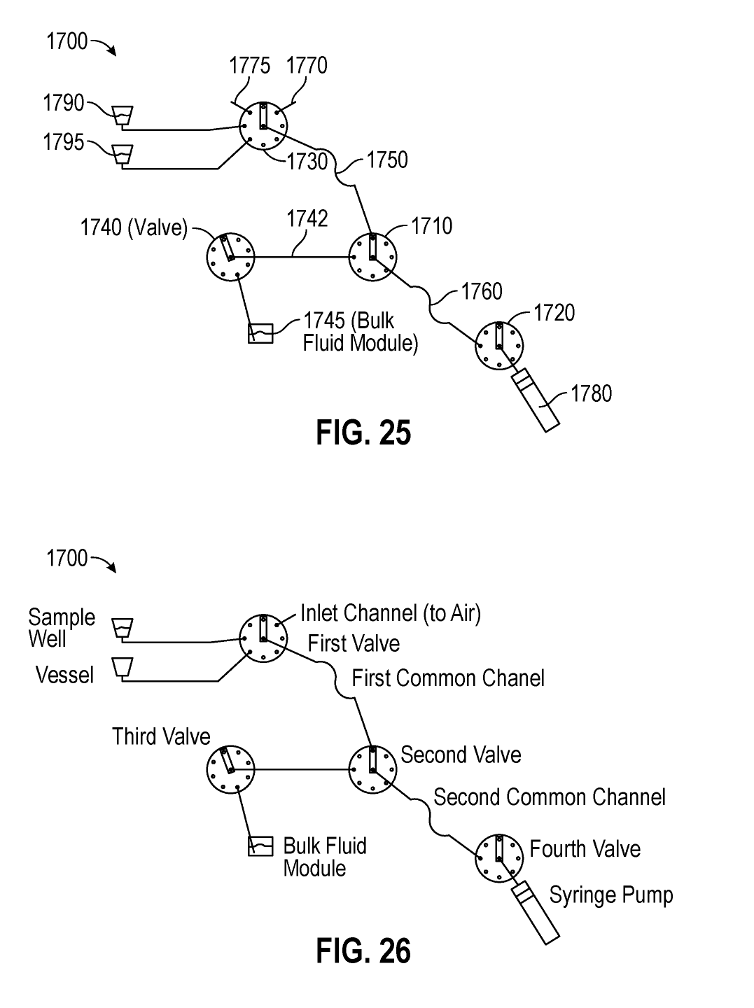

Exemplary channel system 1700 is shown in FIGS. 25-26.

1) Actuate a valve 1710 such that a first common microfluidic channel 1750 and a second common microfluidic channel 1760 are in fluidic communication

2) Actuate a valve 1720 such that second common microfluidic channel 1760 and a syringe pump 1780 are in fluidic communication.

3) Optionally, actuate a valve 1730 such that first common microfluidic channel 1750 and an inlet channel 1770 are in fluidic communication.

4) Optionally, if step 3 is performed, aspirate with syringe pump 1780 such that air is drawn into first common microfluidic channel 1750.

5) Actuate first valve 1730 such that first common microfluidic channel 1750 and a sample well 1790 are in fluidic communication.

6) Aspirate with syringe pump 1780 such that sample fluid in sample well 1790 is drawn into first common microfluidic channel 1750.

7) Actuate valve 1730 such that first common microfluidic channel 1750 and a vessel 1795 are in fluidic communication.

8) Dispense sample fluid into vessel by operating syringe pump.

### Example 2

The following example demonstrates the operation of a channel system such that a bulk fluid is transferred to a vessel of the channel system.

Exemplary channel system 1700 is shown in FIGS. 25-26.

1) Actuate a valve 1710 such that a second common microfluidic channel 1760 and a microfluidic channel 1742 are in fluidic communication

2) Actuate a valve 1740 such that microfluidic channel 1742 and bulk fluid vessel 1745 are in fluidic communication

3) Aspirate with syringe pump 1780 such that a bulk fluid from bulk fluid vessel 1745 is drawn into second common microfluidic channel 1760.

4) Actuate valve 1710 such that first common microfluidic channel 1750 and second microfluidic channel 1760 are in fluidic communication

5) Actuate valve 1730 such that first common microfluidic channel 1750 and waste channel 1775 are in fluidic communication.

6) Aspirate with syringe pump 1780 such that the bulk fluid in second common microfluidic channel 1760 is drawn into first common microfluidic channel 1750 and such that the leading edge of the bulk fluid enters waste channel 1775 (e.g., such that any fluid that is not the bulk fluid is drawn into the waste channel)

7) Optionally, actuate valve 1730 such that first common microfluidic channel 1750 and inlet channel 1770 are in fluidic communication, and aspirate with syringe pump 1780 such that air is drawing into first common microfluidic channel 1750

8) Actuate valve 1730 such that first common microfluidic channel 1750 and a vessel 1795 are in fluidic communication.

9) Dispense bulk fluid into vessel by operating syringe pump.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed:

1. A cartridge, comprising:
a common microfluidic channel;
a sample inlet connected to a sample channel;
a first vessel connected to a first vessel channel;
a second vessel connected to a second vessel channel;
a first valve; and
a second valve;
wherein each of the common channel, sample channel, first vessel channel and second vessel channel extend from the first valve, and
wherein the common microfluidic channel is positioned between the first valve and the second valve.

2. A cartridge, comprising:
a first set of vessels;
a first valve;
a first set of vessel channels connected to the first set of vessels, wherein each of the channels from the first set of vessel channels is connected to the first valve;
a second set of vessel channels; and
a common microfluidic channel positioned between the first set of vessel channels and the second set of vessel channels.

3. A cartridge as in any one of the preceding claims, wherein the cartridge further comprises a frame comprising a first opening constructed and arranged to house a first cassette and a second opening to house a second cassette.

4. A cartridge as in any one of the preceding claims, wherein the common microfluidic channel is a part of a channel system adjacent to and non-integral to the frame.

5. A cartridge as in any one of the preceding claims, wherein the cartridge further comprises a stored liquid reagent contained in at least one of the first vessel or first set of vessels.

6. A cartridge as in any one of the preceding claims, wherein the vessel(s) containing the stored liquid reagent is/are sealed so as to reduce or prevent evaporation of the stored liquid reagent.

7. A cartridge as in any one of the preceding claims, wherein the cartridge further comprises a stored substantially dry reagent.

8. A cartridge as in any one of the preceding claims, wherein the stored reagents comprise reagents for conducting a first PCR reaction.

9. A cartridge as in any one of the preceding claims, wherein the cartridge interfaces with a lid that can be temperature-controlled.

10. A cartridge as in any one of the preceding claims, wherein the lid covers the vessels.

11. A cartridge as in any one of the preceding claims, wherein the lid forms a top portion of the vessel.

12. A cartridge as in any one of the preceding claims, wherein the heated lid is translucent or transparent.

13. A cartridge as in any one of the preceding claims, wherein the heated lid is configured to allow optical measurements to be taken therethrough.

14. A cartridge as in any one of the preceding claims, wherein the cartridge is in communication with a temperature control device.

15. A cartridge as in any one of the preceding claims, wherein the temperature control device is configured to apply a first temperature to a first cassette and a second temperature to a second cassette.

16. A cartridge as in any one of the preceding claims, wherein the temperature control device comprises one or more thermal pads, thermoelectric components, and/or thermistors.

17. A cartridge as in any one of the preceding claims, wherein the cartridge comprises one or more puncture components constructed and arranged to puncture one or more portions of the cassette upon insertion of the cassette into the cartridge.

18. A cartridge as in any one of the preceding claims, wherein the first cassette and/or second cassette comprises a set of vessels.

19. A cartridge as in any one of the preceding claims, wherein the cartridge comprises a third cassette.

20. A cartridge as in any one of the preceding claims, wherein the first cassette is configured to be positioned in a first opening of the frame.

21. A cartridge as in any one of the preceding claims, wherein the second cassette is configured to be position in a second opening of the frame.

22. A cartridge as in any one of the preceding claims, wherein the third cassette comprises a set of vessels.

23. A cartridge as in any one of the preceding claims, wherein vessels in the set of vessels are not in fluid communication with each other prior their insertion into the cartridge.

24. A cartridge as in any one of the preceding claims, wherein at least one of the cassettes comprises a reagent stored therein.

25. A cartridge as in any one of the preceding claims, wherein at least one of the cassettes is sealed and wherein at least a portion of the vessels in the cassette contains a reagent stored therein.

26. A cartridge as in any one of the preceding claims, wherein at least one of the cassettes contains a first reagent stored therein and a second reagent stored therein, and wherein the first and second reagents are not in fluid communication with one another prior to insertion of the cassette into the cartridge.

27. A cartridge as in any one of the preceding claims, wherein at least one of the cassettes in not in fluid communication with the channel system prior to insertion of the cassette into the cartridge.

28. A cartridge as in any one of the preceding claims, wherein at least one of the cassettes is positioned in the cartridge such that a first reagent and/or a second reagent stored therein are in fluid communication with at least one channel of the channel system.

29. A cartridge as in any one of the preceding claims, wherein at least one of the cassettes and/or set of vessels contains a set of lyospheres.

30. A cartridge as in any one of the preceding claims, wherein at least one of the vessels contains a single lyosphere.

31. A cartridge as in any one of the preceding claims, wherein at least one of the vessels contains two or more lyospheres.

32. A cartridge as in any one of the preceding claims, wherein at least one of the cassettes and/or set of vessels contains a set of primers.

33. A cartridge as in any one of the preceding claims, wherein at least one of the cassettes and/or set of vessels contains a buffer, a wash reagent, and/or an alcohol.

34. A cartridge as in any one of the preceding claims, wherein at least one of the cassettes and/or set of vessels is constructed and arranged to be heated.

35. A cartridge as in any one of the preceding claims, wherein at least one of the cassettes comprises a microfluidic channel.

36. A cartridge as in any one of the preceding claims, wherein at least one of the cassettes is refillable.

37. A cartridge as in any one of the preceding claims, wherein at least one of the cassettes is irreversibly attached to the frame and/or the cartridge.

38. A cartridge as in any one of the preceding claims, wherein at least one of the cassettes has a total working volume of at least about 0.1 mL and less than or equal to 25 mL.

39. A cartridge as in any one of the preceding claims, wherein at least one of the cassettes is a waste cassette.

40. A cartridge as in any one of the preceding claims, wherein the waste cassette has a volume of at least 1 mL and less than or equal to 30 mL.

41. A cartridge as in any one of the preceding claims, wherein the cartridge comprises a first cassette comprising a first set of vessels containing stored lyospheres, a second cassette comprising a second first set of vessels containing stored lyospheres, wherein the first and second cassettes are not in fluid communication with one another.

42. A cartridge as in any one of the preceding claims, wherein the first and second cassettes are constructed and arranged to be heated independently.

43. A cartridge as in any one of the preceding claims, wherein the first cassette is constructed and arranged for conducting a first PCR reaction and the second cassette is constructed and arranged for conducting a second PCR reaction independent of the first PCR reaction.

44. A cartridge as in any one of the preceding claims, wherein the cartridge comprises a channel system.

45. A cartridge as in any one of the preceding claims, wherein the channel system comprises a first set of channels for conducting a first PCR reaction.

46. A cartridge as in any one of the preceding claims, wherein the channel system comprises a second set of channels for conducting a second PCR reaction.

47. A cartridge as in any one of the preceding claims, wherein the first and second set of channels are separated by at least one valve.

48. A cartridge as in any one of the preceding claims, wherein the common channel comprises a serpentine channel.

49. A cartridge as in any one of the preceding claims, wherein an internal volume of the first vessel channel is less than an internal volume of the second vessel channel.

50. A cartridge as in any one of the preceding claims, wherein the first set of vessel channels and/or second set of vessel channels includes at least 2, 4, 6, 8, or 10 channels and/or less than or equal to 20, 15, 10, or 5 channels.

51. A cartridge as in any one of the preceding claims, wherein the common channel has a volume of at least 2 μL and/or less than or equal to 200 μL.

52. A cartridge as in any one of the preceding claims, wherein the channel system comprises a second set of vessels connected to the second set of vessel channels.

53. A cartridge as in any one of the preceding claims, wherein the channel system comprises a second valve, wherein each of the channels from the second set of vessel channels is connected to the second valve.

54. A cartridge as in any one of the preceding claims, wherein the common microfluidic channel, the first vessel channel, and/or the second vessel channel have a largest cross-sectional dimension of greater than or equal to about 50 microns and/or less than or equal to 1 mm.

55. A cartridge as in any one of the preceding claims, wherein each vessel has a volume of at least about 1 μL and/or less than or equal to 200 μL.

56. A cartridge as in any one of the preceding claims, wherein the first set of vessels and/or second set of vessels includes at least 2, 4, 6, 8, or 10 vessels and/or less than or equal to 20, 15, 10, or 5 vessels.

57. A cartridge as in any one of the preceding claims, wherein at least one vessel has a tapered cross-sectional shape.

58. A cartridge as in any one of the preceding claims, wherein at least one vessel is conical in shape.

59. A cartridge as in any one of the preceding claims, wherein the frame and/or cartridge is in direct contact with a carrier plate.

60. A cartridge as in any one of the preceding claims, wherein the carrier plate is configured to facilitate transport of the frame and/or cartridge without a user containing the frame and/or cartridge.

61. A cartridge as in any one of the preceding claims, wherein the frame comprises one or more sample wells configured to receive one or more samples.

62. A cartridge as in any one of the preceding claims, wherein the one or more sample wells are in fluidic communication with the channel system.

63. A cartridge as in any one of the preceding claims, wherein the frame comprises one or more output wells.

64. A cartridge as in any one of the preceding claims, wherein the one or more output wells are in fluidic communication with the channel system.

65. A cartridge as in any one of the preceding claims, wherein the frame comprises a waste cassette.

66. A cartridge as in any one of the preceding claims, wherein the first valve and/or second valve is a rotary valve.

67. A cartridge as in any one of the preceding claims, wherein the first valve and/or second valve comprises a raised feature configured to facilitate the flow of a fluid between the common microfluidic channel and another channel.

68. A cartridge as in any one of the preceding claims, wherein the valve comprises a seal.

69. A cartridge as in any one of the preceding claims, wherein the channel system comprises a waste channel connected to a waste vessel.

70. A method, comprising:
flowing, in a first direction, a first fluid in a common microfluidic channel;
flowing at least a portion of the first fluid in the common microfluidic channel in a second direction, wherein the second direction is opposite the first direction;
flowing at least a portion of the first fluid into a first vessel via a first vessel channel;
flowing at least a portion of the first fluid from the first vessel to the common microfluidic channel; and
flowing at least a portion of the first fluid from the common channel to a second vessel via a second vessel channel.

71. A method, comprising:
flowing a first fluid in a common microfluidic channel;
flowing a portion of the first fluid into a first vessel;
flowing a portion of the first fluid into a waste vessel;
performing a chemical and/or biological reaction in the first vessel to form a second fluid;
flowing a portion of the second fluid from the first vessel to the common microfluidic channel; and
flowing a portion of the second fluid into the waste vessel.

72. A method, comprising:
flowing a first fluid into a common microfluidic channel;
actuating a valve such that the common microfluidic channel is in fluidic communication with a first vessel channel;
flowing at least a portion of the first fluid into the first vessel channel;
introducing a second fluid into the common microfluidic channel, wherein the second fluid is immiscible with the first fluid;
flowing at least a portion of the second fluid from the common microfluidic channel into the first vessel channel; and
flowing a controlled volume of the first fluid into a first vessel connected to the first vessel channel during flow of the second in the first vessel channel.

73. A method as in any one of the preceding claims, wherein the cartridge comprises a channel system.

74. A method as in any one of the preceding claims, wherein the channel system comprises a first set of channels for conducting a first PCR reaction.

75. A method as in any one of the preceding claims, wherein the channel system comprises a second set of channels for conducting a second PCR reaction.

76. A method as in any one of the preceding claims, wherein the first and second set of channels are separated by at least one valve.

77. A method as in any one of the preceding claims, wherein the common channel comprises a serpentine channel.

78. A method as in any one of the preceding claims, wherein an internal volume of the first vessel channel is less than an internal volume of the second vessel channel.

79. A method as in any one of the preceding claims, wherein the first set of vessel channels and/or second set of vessel channels includes at least 2, 4, 6, 8, or 10 channels and/or less than or equal to 20, 15, 10, or 5 channels.

80. A method as in any one of the preceding claims, wherein the common channel has a volume of at least 2 μL and/or less than or equal to 200 μL.

81. A method as in any one of the preceding claims, wherein the channel system comprises a second set of vessels connected to the second set of vessel channels.

82. A method as in any one of the preceding claims, wherein the channel system comprises a second valve, wherein each of the channels from the second set of vessel channels is connected to the second valve.

83. A method as in any one of the preceding claims, wherein the common microfluidic channel, the first vessel channel, and/or the second vessel channel have a largest cross-sectional dimension of greater than or equal to about 50 microns and/or less than or equal to 1 mm.

84. A method as in any one of the preceding claims, wherein, upon entering the first vessel, the fluid is exposed to a first reagent.

85. A method as in any one of the preceding claims, wherein, upon entering the second vessel, the fluid is exposed to a second reagent.

86. A method as in any one of the preceding claims, wherein a sample and/or reactant present in the fluid reacts with the first reagent.

87. A method as in any one of the preceding claims, wherein the sample and/or the reactant present in the fluid reacts with the second reagent.

88. A method as in any one of the preceding claims, wherein flowing the first fluid comprises actuating a valve such that the common microfluidic channel is in fluidic communication with a first vessel channel.

89. A method as in any one of the preceding claims, wherein flowing the first fluid comprises applying a second pressure to the common microfluidic channel such that at least a portion of a second fluid enters the vessel channel.

90. A method as in any one of the preceding claims, wherein a volume of the fluid present in the vessel channel and/or a vessel connected to the vessel path is controlled by the first and/or second pressure applied to the common microfluidic channel.

91. A method as in any one of the preceding claims, wherein each of the common channel and vessel channel(s) extend from the valve.

92. A method as in any one of the preceding claims, wherein flowing at least a portion of the first fluid comprises actuating a/the valve such that the common microfluidic channel is in fluidic communication with the first vessel channel.

93. A method as in any one of the preceding claims, wherein flowing at least a portion of the first fluid into the first vessel via the first vessel channel comprises applying a first pressure to the common microfluidic channel.

94. A method as in any one of the preceding claims, wherein flowing at least a portion of the first fluid from the first vessel to the common microfluidic channel comprises applying a second pressure to the common microfluidic channel.

95. A method as in any one of the preceding claims, wherein flowing at least a portion of the first fluid from the common channel to the second vessel via the second vessel channel comprises actuating the valve such that the common microfluidic channel is in fluidic communication with the second vessel channel.

96. A method as in any one of the preceding claims, wherein flowing at least a portion of the first fluid from the common channel to the second vessel via the second vessel channel comprises applying a third pressure to the microfluidic channel.

97. A method as in any one of the preceding claims, wherein flowing at least a portion of the second fluid comprises applying a pressure to the microfluidic channel.

98. A method as in any one of the preceding claims, wherein a pressure (the first pressure, the second pressure, and/or the third pressure) is a positive pressure.

99. A method as in any one of the preceding claims, wherein a pressure (the first pressure, the second pressure, and/or the third pressure) is a negative or reduced pressure.

100. A method as in any one of the preceding claims, wherein flowing at least a portion of the second fluid comprises actuating the valve such that the common microfluidic channel is in fluidic communication with the first vessel channel

101. A method as in any one of the preceding claims, wherein flowing at least a portion of the second fluid from the common microfluidic channel into the first vessel channel comprises preventing the second fluid from entering into the first vessel.

102. A method as in any one of the preceding claims, wherein introducing the second fluid into the common microfluidic channel comprises flowing the second fluid through a sample inlet connected to a sample channel extending from the valve and in fluidic communication with the common microfluidic channel.

103. A method as in any one of the preceding claims, wherein the controlled volume has a volume of less than or equal to less than or equal to 100 μL, less than or equal to 80 μL, less than or equal to 60 μL, less than or equal to 40 μL, less than or equal to 20 μL, less than or equal to 10 μL, less than or equal to 5 μL, or less than or equal to 2 μL.

104. A method as in any one of the preceding claims, wherein the first fluid is a liquid sample

105. A method as in any one of the preceding claims, wherein the first fluid is a reagent.

106. A method as in any one of the preceding claims, wherein the second fluid is a gas.

107. A method as in any one of the preceding claims, wherein the gas is air.

108. A method as in any one of the preceding claims, wherein the second fluid is a reagent.

109. A method as in any one of the preceding claims, wherein the second fluid is a liquid sample.

110. A method as in any one of the preceding claims, wherein the second fluid is water or a buffer.
